(19) United States
(12) Patent Application Publication
(10) Pub. No.: US 2022/0068490 A1
(43) Pub. Date: Mar. 3, 2022

(54) TEST APPARATUS, TEST METHOD, AND TEST PROGRAM

(71) Applicant: FUJIFILM CORPORATION, Tokyo (JP)

(72) Inventors: Haruyasu NAKATSUGAWA, Kanagawa (JP); Yasuhisa KANEKO, Kanagawa (JP); Kenji NAGAMIYA, Kanagawa (JP); Tomohide HIRAGAMI, Kanagawa (JP); Yasuyuki HOSONO, Kanagawa (JP); Nobuya KITAMURA, Kanagawa (JP)

(21) Appl. No.: 17/409,808

(22) Filed: Aug. 24, 2021

(30) Foreign Application Priority Data
Aug. 25, 2020 (JP) 2020-142090
Jan. 12, 2021 (JP) 2021-002929
May 27, 2021 (JP) 2021-089606

(51) Int. Cl. G16H 50/30; A61B 5/0537; A61B 5/00; A61B 5/145

(52) U.S. Cl. CPC G16H 50/30; A61B 5/0537; A61B 5/681; A61B 5/0013; A61B 5/7445; A61B 5/0022; A61B 5/14532

(57) ABSTRACT

A test apparatus comprising at least one processor, wherein the processor is configured to acquire a determination result as to whether or not there is an abnormal tendency in first biological information, the determination result being determined based on a monitoring result of the first biological information of a user, determine, as a test candidate to be recommended for the user, at least one test candidate for acquiring second biological information associated with the first biological information in a case where the determination result corresponds to the abnormal tendency, present the determined test candidate to the user, receive selection of the presented test candidate, and present a guide according to the selected test candidate to the user.

### CROSS-REFERENCE TO RELATED APPLICATIONS

The present application claims priority under 35 U.S.C. § 119 to Japanese Patent Application No. 2020-142090, filed on Aug. 25, 2020, Japanese Patent Application No. 2021-002929, filed on Jan. 12, 2021, and Japanese Patent Application No. 2021-089606, filed on May 27, 2021, the entire disclosures of which are incorporated by reference herein.

### BACKGROUND

### Technical Field

The technique of the present disclosure relates to a test apparatus, a test method, and a test program.

### Related Art

In recent years, a method of monitoring biological information such as a pulse, a blood pressure, respiration, an electrocardiogram, maximum oxygen intake, a blood glucose equivalent value, and a body temperature by using a wearable terminal such as a smart watch and managing health improvement and disease prevention based on the monitored biological information is beginning to spread. In addition, by learning artificial intelligence (AI) using big data related to biological information, it is possible to identify diseases and potential diseases with high accuracy.

Further, a method of providing health management information to a user using biological information has been proposed. For example, JP2006-259827A proposes a method of presenting a recommended action plan such as content of an exercise and content of a meal in a case where an abnormality is observed in a monitoring result of a blood glucose value acquired by a wearable terminal.

On the other hand, JP2012-068155A proposes an automatic test apparatus that automatically performs a biological test. The automatic test apparatus described in JP2012-068155A is provided such that a test kit can be removed, and a user collects a sample such as blood using the test kit. In a case where the user sets the sample in the automatic test apparatus, the automatic test apparatus performs a test using the sample and transmits a test result to a personal computer or a mobile terminal of the user. In a case where the automatic test apparatus is provided in a company, a pharmacy, a convenience store, or the like, the user can perform a test at a desired timing without going to hospital.

### SUMMARY

On the other hand, a wearable terminal can monitor biological information. However, accuracy of the acquired biological information is lower than accuracy of biological information based on a blood test or the like. In the method described in JP2006-259827A, only a recommended action plan is presented to the user. For this reason, in the method described in JP2006-259827A, it is difficult to recognize a current state of biological information with high accuracy in a case where an abnormal tendency is observed in the monitoring result. Further, in the method described in JP2012-068155A, the user voluntarily performs a specific test using a test kit, and a test based on biological information acquired by a wearable terminal or the like is not performed.

The present disclosure has been made in view of the above circumstances, and an object of the present disclosure is to allow a user to perform a desired test by using a monitoring result of biological information.

According to an aspect of the present disclosure, there is provided a test apparatus including: at least one processor configured to acquire a determination result as to whether or not there is an abnormal tendency in first biological information, the determination result being determined based on a monitoring result of the first biological information of a user, determine, as a test candidate to be recommended for the user, at least one test candidate for acquiring second biological information associated with the first biological information in a case where the determination result corresponds to the abnormal tendency, present the determined test candidate to the user, receive selection of the presented test candidate; and present a guide according to the selected test candidate to the user.

In the test apparatus according to the aspect of the present disclosure, the processor may be configured to present the test candidate for acquiring the second biological information by collecting a sample of the user, present, to the user, a guide for collecting the sample in a case where the test candidate is selected, and acquire the second biological information based on the sample collected by the user.

In the test apparatus according to the aspect of the present disclosure, the processor may be configured to present the second biological information to the user.

The test apparatus according to the aspect of the present disclosure may further include an analysis apparatus that obtains the second biological information by analyzing the collected sample.

In the test apparatus according to the aspect of the present disclosure, the processor may be configured to present the test candidate for acquiring the second biological information by delivering a sample of the user to an external test agency, and present, to the user, a guide for a procedure of delivering the first biological information of the user to the user in a case where the test candidate is selected.

The test apparatus according to the aspect of the present disclosure may further include a container case that contains a test kit required for performing a test according to the test candidate to be presented.

In the test apparatus according to the aspect of the present disclosure, the test candidate may include a test candidate in which the second biological information is acquired with accuracy higher than accuracy of the first biological information.

In the test apparatus according to the aspect of the present disclosure, the processor may be configured to receive designation of at least one item serving as an index in a case where the user selects the presented test candidate, and present the test candidate in a display form according to the designated item.

In the test apparatus according to the aspect of the present disclosure, the processor may be configured to present, to the user, a question for determining a display position of the test candidate on at least one scale according to the designated at least one item, determine the display position of the test candidate on the scale according to an answer to the question, and present, to the user, the test candidate in a display form in which an icon representing the test candidate is displayed at the determined display position on the scale.

In the test apparatus according to the aspect of the present disclosure, the processor may be configured to receive additional designation of the item by the user after the icon representing the test candidate is displayed, and update the display form of the icon representing the test candidate in a case where designation of the item is added.

In the test apparatus according to the aspect of the present disclosure, the processor may be configured to repeatedly perform receiving of additional designation of the item, presenting of a question according to the added item, and updating of the display form of the test candidate until the test candidate is selected by the user.

In the test apparatus according to the aspect of the present disclosure, the item may include at least one of a cost required for a test, a sense of stability in a case where a sample is collected, a degree of restraint in a test, invasiveness or non-invasiveness, a time required to obtain a test result, whether to include a risk check for various diseases, or test accuracy.

In the test apparatus according to the aspect of the present disclosure, the processor may be configured to selectively determine the test candidate from plural test methods including designation of a type of a sample and used for acquiring the second biological information from the sample.

In the test apparatus according to the aspect of the present disclosure, the processor may be configured to determine the test candidate according to a degree of the abnormal tendency.

In the test apparatus according to the aspect of the present disclosure, the processor may be configured to determine the test candidate according to an elapsed time from a time when it is determined that there is the abnormal tendency in the first biological information to a current time.

In the test apparatus according to the aspect of the present disclosure, the processor may be configured to determine the test candidate according to a risk factor that the user has.

In the test apparatus according to the aspect of the present disclosure, the processor may be configured to present, as the guide, support information for supporting execution of a test of the selected test candidate, the support information being transmitted in real time from a remote location.

The test apparatus according to the aspect may further include a camera that captures an image of the user. Further, the processor may be configured to transmit, to the remote location, a moving image obtained by capturing a state where the user performs the test of the selected test candidate by the camera.

In the test apparatus according to the aspect of the present disclosure, the processor may be configured to present, to the user, statistical information of the test candidate which is previously selected.

In the test apparatus according to the aspect of the present disclosure, the first biological information may be a blood glucose equivalent value that correlates with a blood glucose value, and the second biological information may include at least one of a blood glucose value or HbA1c.

In the test apparatus according to the aspect of the present disclosure, the abnormal tendency may be determined based on a postprandial hyperglycemic spike.

In the test apparatus according to the aspect of the present disclosure, the processor may be configured to determine the test candidate based on meal content of the user.

In the test apparatus according to the aspect of the present disclosure, the processor may be configured to determine the test candidate based on a meal time of the user.

In the test apparatus according to the aspect of the present disclosure, the processor may be configured to present the test candidate including an action of drinking a glucose drink in a case where a predetermined time has elapsed from a time after meal based on the meal time.

In the test apparatus according to the aspect of the present disclosure, the first biological information may be at least one of a heart rate, a blood pressure, respiration, an electrocardiogram, maximum oxygen intake, arterial oxygen saturation, or a body temperature, and the second biological information may be a diagnosis result of a disease of the user.

In the test apparatus according to the aspect of the present disclosure, the processor may be configured to notify the user of a fact that there is no abnormal tendency in the first biological information in a case where a determination result indicating the fact is acquired.

In the test apparatus according to the aspect of the present disclosure, the first biological information may be monitored by a measurement device that the user wears, and the processor may be configured to acquire the monitoring result of the first biological information from the measurement device, and acquire the determination result by determining whether or not there is the abnormal tendency in the first biological information based on the acquired monitoring result of the first biological information.

In the test apparatus according to the aspect of the present disclosure, the determination result may be obtained by a measurement device attached to the user, and the processor may be configured to acquire the determination result from the measurement device.

In this case, the measurement device may be a wearable measurement device.

In the test apparatus according to the aspect of the present disclosure, the processor may be configured to perform a notification for urging the user to wear the measurement device for a longer time in a case where whether or not there is the abnormal tendency in the first biological information is not determined.

In the test apparatus according to the aspect of the present disclosure, the processor may be configured to acquire the second biological information, and determine whether to continue monitoring of the first biological information according to the acquired second biological information.

According to another aspect of the present disclosure, there is provided a test method including: acquiring a determination result as to whether or not there is an abnormal tendency in first biological information, the determination result being determined based on a monitoring result of the first biological information of a user; determining, as a test candidate to be recommended for the user, at least one test candidate for acquiring second biological information associated with the first biological information in a case where the determination result corresponds to the abnormal tendency; presenting the determined test candidate to the user; receiving selection of the presented test candidate; and presenting a guide according to the selected test candidate to the user.

According to still another aspect of the present disclosure, there is provided a test program causing a computer to execute: a procedure of acquiring a determination result as to whether or not there is an abnormal tendency in first biological information, the determination result being determined based on a monitoring result of the first biological information of a user; a procedure of determining, as a test candidate to be recommended for the user, at least one test candidate for acquiring second biological information associated with the first biological information in a case where the determination result corresponds to the abnormal tendency; a procedure of presenting the determined test candidate to the user; a procedure of receiving selection of the presented test candidate; and a procedure of presenting a guide according to the selected test candidate to the user.

According to the present disclosure, the user can perform a test according to a monitoring result of biological information.

### BRIEF DESCRIPTION OF THE DRAWINGS

FIG. 1 is a schematic diagram illustrating a configuration of a test support system to which a test apparatus according to an embodiment of the present disclosure is applied.

FIG. 2 is a hardware configuration diagram of the test apparatus according to the present embodiment.

FIG. 3 is a hardware configuration diagram of a mobile terminal.

FIG. 4 is a hardware configuration diagram of a wristwatch-type measurement device.

FIG. 5 is a hardware configuration diagram of the measurement device for measuring glucose in an interstitial fluid.

FIG. 6 is a functional configuration diagram of the test apparatus according to the present embodiment.

FIG. 7 is a graph illustrating daily variations in blood glucose of a patient with type 2 diabetes.

FIG. 8 is a table illustrating test methods for biological information associated with a blood glucose equivalent value.

FIG. 9 is a flowchart illustrating processing performed in the present embodiment.

FIG. 10 is a flowchart illustrating processing performed in the present embodiment.

FIG. 11 is a diagram illustrating a notification screen in a case where there is no abnormal tendency in the blood glucose equivalent value.

FIG. 12 is a diagram illustrating a test candidate presentation screen.

FIG. 13 is a diagram illustrating a test candidate presentation screen.

FIG. 14 is a diagram illustrating a test candidate presentation screen.

FIG. 15 is a diagram illustrating a test candidate presentation screen.

FIG. 16 is a diagram illustrating a test candidate presentation screen on which a description of a selected test candidate is displayed.

FIG. 17 is a diagram illustrating a test candidate presentation screen on which statistical information of a test candidate which is previously selected is displayed.

FIG. 18 is a diagram illustrating an item list screen including an item serving as an index in a case where a user selects a test candidate.

FIG. 19 is a diagram illustrating a question presentation screen.

FIG. 20 is a diagram illustrating a presentation screen for presenting test candidates to a user in a display form in which test candidate icons are displayed on a scale.

FIG. 21 is a diagram illustrating a presentation screen for presenting test candidates to a user in a display form in which test candidate icons are displayed on a scale.

FIG. 22 is a diagram illustrating an item list screen including an item serving as an index in a case where a user selects a test candidate.

FIG. 23 is a diagram illustrating a question presentation screen.

FIG. 24 is a diagram illustrating a presentation screen for presenting test candidates to a user in a display form in which test candidate icons are displayed on a scale in a case where two items are selected.

FIG. 25 is a diagram illustrating a guide screen for reservation of a test.

FIG. 26 is a diagram illustrating an analysis result display screen.

FIG. 27 is a diagram illustrating a guide screen for guiding a website that sells a blood glucose measurement device.

FIG. 28 is a diagram illustrating a guide screen for guiding, to a user, a hospital at which a glucose tolerance test can be performed.

FIG. 29 is a diagram illustrating a guide screen for a next test.

FIG. 30 is a diagram illustrating a notification screen for urging a user to wear a measurement device for a longer time.

FIG. 31 is a graph illustrating a comparison result between a monitoring result of a blood glucose equivalent value and a test result obtained by blood sampling of a user.

FIG. 32 is a diagram illustrating a guide screen for a delivery procedure.

FIG. 33 is a schematic graph illustrating transitions in heart rate variability and the like of a patient who is infected with new coronavirus infection.

FIG. 34 is a table illustrating test methods related to new coronavirus infection.

FIG. 35 is a diagram illustrating a presentation screen for presenting test candidates to a user in a display form in which test candidate icons are displayed on a scale in a case where two items are selected.

FIG. 36 is a diagram illustrating a presentation screen for presenting test candidates to a user in a display form in which test candidate icons are displayed on a scale in a case where two items are selected.

FIG. 37 is a diagram illustrating a question presentation screen.

### DESCRIPTION OF EMBODIMENTS

### First Embodiment

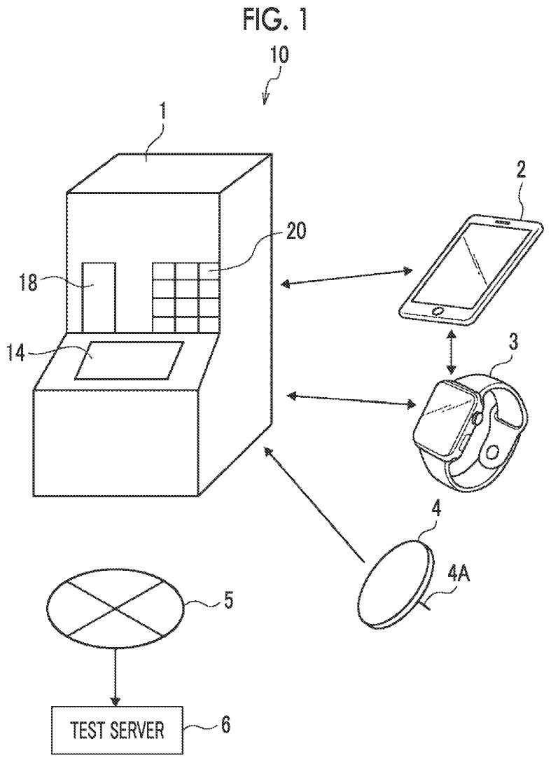

Hereinafter, a first embodiment of the present disclosure will be described with reference to the drawings. FIG. 1 is a schematic diagram illustrating a configuration of a test support system to which a test apparatus according to a first embodiment of the present disclosure is applied. As illustrated in FIG. 1, a test support system 10 includes a test apparatus 1 according to the present embodiment, a mobile terminal 2 such as a smartphone, and measurement devices 3 and 4. The mobile terminal 2 and the measurement devices 3 and 4 can communicate with each other by short-range wireless communication such as Bluetooth (registered trademark). In addition, the test apparatus 1 and the measurement devices 3 and 4 can also communicate with each other by short-range wireless communication. Further, the test apparatus 1 and the mobile terminal 2 can also communicate with each other by short-range wireless communication. The test apparatus 1 is connected to a test server 6 via a wired/wireless network 5 such that communication can be performed.

The test apparatus 1 is provided in a place at which people gather, such as a convenience store, a shopping mall, a station, an airport, a company, a pharmacy, a public hall, a care plaza for aged persons, and a hot spring facility. Alternatively, the test apparatus 1 is provided in a moving vehicle, and is moved to a desired place and is used. The test apparatus 1 determines whether or not there is an abnormal tendency in a blood glucose equivalent value of a user based on a monitoring result of the blood glucose equivalent value transmitted from the measurement device 3 or the like by the user, and presents a test candidate for acquiring biological information associated with the blood glucose equivalent value to the user according to a determination result. In addition, the test apparatus 1 assists a test to be performed by the user, by presenting, to the user, a guide according to the test candidate selected by the user. The blood glucose equivalent value is biological information that correlates with the blood glucose value and is measured by a method that does not rely on blood sampling. Further, the test apparatus 1 illustrated in FIG. 1 includes an analysis apparatus 18 and a container case 20, which will be described.

The measurement device 3 is a wristwatch-type wearable terminal such as a smart watch, and has a function of monitoring a blood glucose equivalent value that correlates with a blood glucose value of a user. The monitoring means that the blood glucose equivalent value is automatically and constantly measured at a predetermined time interval, for example, 15 minutes, 30 minutes, or the like, without a measurement instruction from a user. The measurement device 3 may measure the blood glucose equivalent value even in a case where an instruction from a user is input while constantly measuring the blood glucose equivalent value. Further, the measurement device 3 may be a device that is worn by the user only at the time of measurement, such as a finger clip.

Further, the measurement device 3 is a non-invasive measurement device for the blood glucose equivalent value, and obtains the blood glucose equivalent value by, for example, irradiating the user with infrared rays and analyzing a signal emitted by glucose in blood. Alternatively, the measurement device 3 measures an electrocardiogram of the user, and obtains a blood glucose equivalent value that correlates with changes in the electrocardiogram. The measurement device 3 transmits the obtained blood glucose equivalent value to the test apparatus 1.

The measurement device 4 is an invasive measurement device for the blood glucose equivalent value. For example, the measurement device 4 is attached to the user, monitors a glucose concentration in an interstitial fluid under epidermis of the user, and transmits the measured glucose concentration to the test apparatus 1. For this reason, the measurement device 4 includes a needle-shaped filament 4A that is inserted under the epidermis of the user. The glucose concentration correlates with the blood glucose value, and thus the glucose concentration corresponds to the blood glucose equivalent value.

The blood glucose equivalent value measured by the measurement devices 3 and 4 is an example of first biological information. The user may possess any one of the measurement device 3 or the measurement device 4.

Here, in the present embodiment, the test apparatus 1 determines whether or not there is an abnormal tendency in the blood glucose equivalent value based on a monitoring result of the blood glucose equivalent value. Specifically, the test apparatus 1 determines whether or not there is an abnormal tendency in the blood glucose equivalent value based on a state of a postprandial hyperglycemic spike. The postprandial hyperglycemic spike is a symptom observed in an early stage of diabetes, and is a symptom in which the blood glucose value significantly increases approximately one hour to two hours after a meal even in a case where a fasting blood glucose value is within a normal range. In a case where the postprandial hyperglycemic spike is neglected, blood vessels are damaged and complications of arteriosclerosis and diabetes are more likely to progress. It is also considered that complications such as myocardial infarction, angina, and stroke are more likely to progress. In the present embodiment, the blood glucose equivalent value is monitored, and in a case where it is determined that there is an abnormal tendency in the blood glucose equivalent value, the test candidate to be recommended for the user is presented to the user. Thereby, it is possible to allow the user to take a test in which biological information for accurately recognizing the postprandial hyperglycemic spike can be acquired.

The network 5 is a wide area network (WAN) that widely connects the test apparatus 1 and the test server 6 via a public line network or a dedicated line network.

The test server 6 is provided in a test center that supports a test related to the blood glucose value. The test server 6 has a function of providing a test program according to the present embodiment to the test apparatus 1 or a function of providing information which is required when the test apparatus 1 executes the test program to the test apparatus 1. In the test server 6, a software program for providing server functions to a general-purpose computer is installed.

The test center provides, to the user, various support for the test related to the blood glucose value. For example, the test center provides a test program according to the present embodiment. Thus, the test center supports the user to select a test method. In addition, in a case where the user selects a test method, the test center also supports purchase of devices and test kits required for the selected test, and supports hospital test reservations. Further, the test center also performs a test using a sample, which is obtained by self blood sampling of a user and is delivered by the user. The test center is an example of an external test agency.

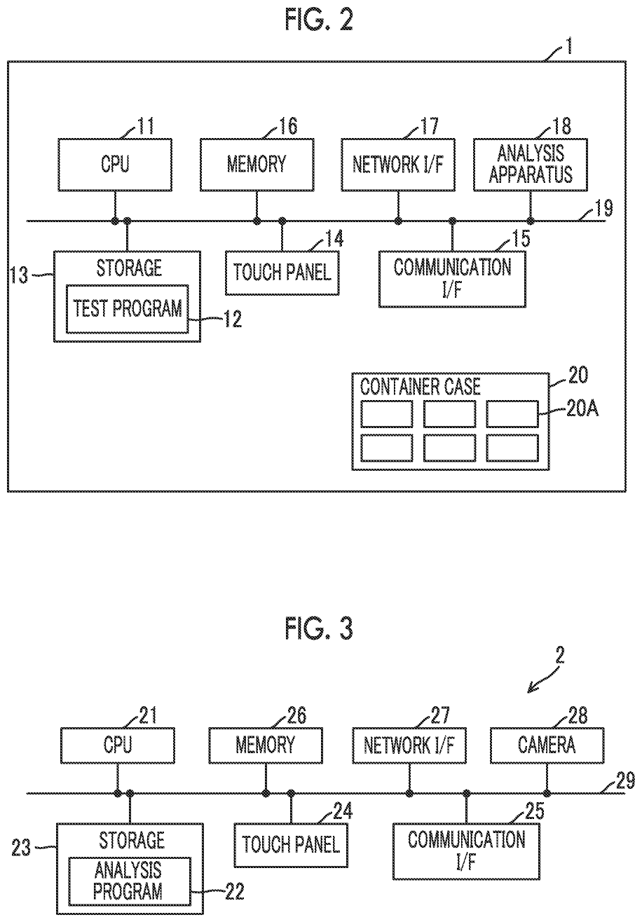

Next, the test apparatus according to the present embodiment will be described. A hardware configuration of the test apparatus according to the present embodiment will be described with reference to FIG. 2. As illustrated in FIG. 2, the test apparatus 1 as a test apparatus according to the present embodiment includes a central processing unit (CPU) 11, a non-volatile storage 13, and a memory 16 as a transitory storage area. Further, the test apparatus 1 includes a touch panel 14, a communication interface (I/F) 15 for short-range wireless communication, and a network I/F 17 wirelessly connected to the network 5. The test apparatus 1 further includes an analysis apparatus 18. The test apparatus 1 further includes a container case 20. The CPU 11, the storage 13, the touch panel 14, the communication I/F 15, the memory 16, the network I/F 17, and the analysis apparatus 18 are connected to a bus 19. The CPU 11 is an example of a processor according to the present disclosure.

The storage 13 is realized by a solid state drive (SSD), a flash memory, or the like. A test program 12 installed in the test apparatus 1 is stored in the storage 13 as a storage medium. The CPU 11 reads out the test program 12 from the storage 13, develops the test program 12 in the memory 16, and executes the developed test program 12.

The touch panel 14 is configured with a liquid crystal display, an organic EL, or the like, and performs various displays related to processing performed by the test apparatus 1. The touch panel 14 also has a function as an input device for inputting various information to the test apparatus 1.

The test program 12 is stored in the test server 6 in a state of being accessible from the outside, and is downloaded and installed in the test apparatus 1 in response to a request.

The analysis apparatus 18 obtains biological information such as a blood glucose value and HbA1c (hemoglobin A1c) by analyzing blood of the user. For this reason, the analysis apparatus 18 includes a mechanism for sampling blood of the user, and a mechanism for acquiring blood from a test kit to be described.

The container case 20 contains plural test kits 20A for obtaining the blood of the user. The test kit 20A includes a blood sampling device and a glucose drink for the user to drink before a test. In a case where the user selects a test including self blood sampling to be described, the user can remove the test kit from the container case, drink a glucose drink if necessary, and then perform self blood sampling using a blood sampling device.

Next, the mobile terminal 2 will be described. FIG. 3 is a hardware configuration diagram of the mobile terminal 2. As described above FIG. 3, the mobile terminal 2 is a portable computer such as a smartphone, and includes a CPU 21, a non-volatile storage 23, and a memory 26 as a transitory storage area. Further, the mobile terminal 2 includes a touch panel 24, a communication interface (I/F) 25 for short-range wireless communication, and a network I/F 27 wirelessly connected to the network 5. Further, the mobile terminal 2 includes a camera 28. The CPU 21, the storage 23, the touch panel 24, the communication I/F 25, the memory 26, the network I/F 27, and the camera 28 are connected to a bus 29.

The storage 23 is realized by a solid state drive (SSD), a flash memory, or the like. An analysis program 22 installed in the mobile terminal 2 is stored in the storage 23 as a storage medium. The CPU 21 reads out the analysis program 22 from the storage 23, develops the analysis program 22 in the memory 26, and executes the developed analysis program 22.

The touch panel 24 is configured with a liquid crystal display, an organic EL, or the like, and performs various displays related to processing performed by the mobile terminal 2. The touch panel 24 also has a function as an input device for inputting various information to the mobile terminal 2.

The camera 28 acquires an image of a meal of the user by capturing the meal of the user according to, for example, an instruction of the user. The acquired image of the meal is stored in the storage 23. Alternatively, as described later, the acquired image of the meal is transmitted to the test apparatus 1 together with the monitoring result of the blood glucose equivalent value according to an instruction of the user or without waiting for an instruction of the user.

The analysis program 22 is stored in the test server 6 in a state of being accessible from the outside, and is downloaded and installed in the mobile terminal 2 in response to a request. The analysis program 22 determines whether or not there is an abnormal tendency in the blood glucose equivalent value by analyzing the monitoring result of the blood glucose equivalent value transmitted from the measurement devices 3 and 4. The analysis program 22 may execute only processing of storing the blood glucose equivalent value transmitted from the measurement devices 3 and 4 in the storage 23. In a case where the monitoring result of the blood glucose equivalent value is transmitted from the measurement devices 3 and 4 to the test apparatus 1, the mobile terminal 2 is not used.

Further, in the present embodiment, it is assumed that user registration to the test center is completed by the user. In a case where the user is authenticated in the test apparatus 1, the monitoring result of the blood glucose equivalent value may be transmitted to the test apparatus 1, or a test may be performed in the test apparatus 1.

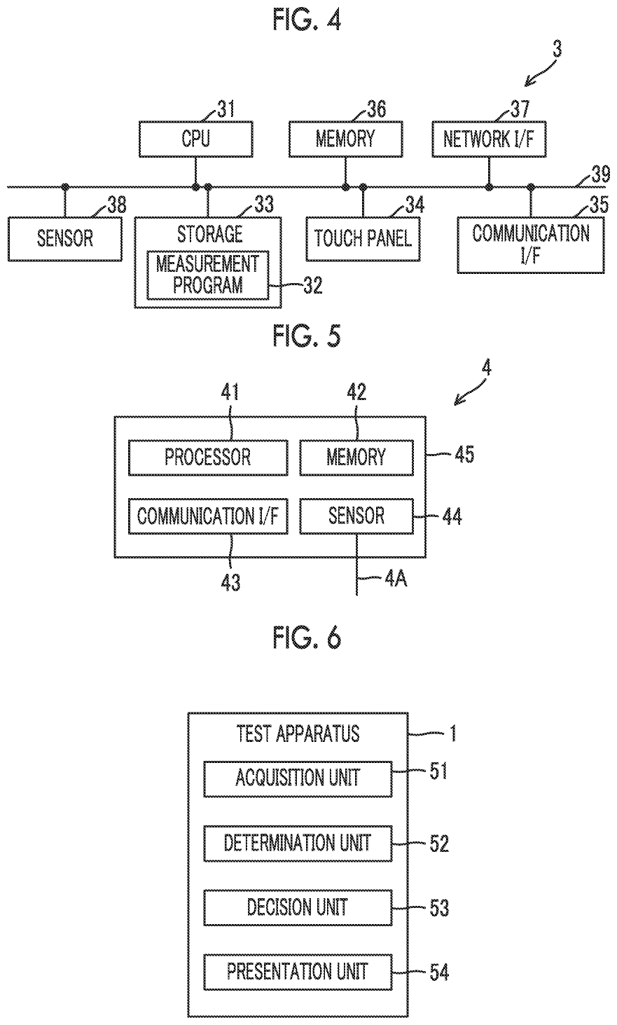

Next, the measurement device will be described. FIG. 4 illustrates a hardware configuration of the measurement device 3. The measurement device 3 is a wristwatch-type computer, and as illustrated in FIG. 4, includes a CPU 31, a non-volatile storage 33, and a memory 36 as a transitory storage area. Further, the measurement device 3 includes a touch panel 34, a communication I/F 35 for short-range wireless communication, a network I/F 37 wirelessly connected to an external network (not illustrated), and a sensor 38. The CPU 31, the storage 33, the touch panel 34, the communication I/F 35, the memory 36, the network I/F 37, and the sensor 38 are connected to a bus 39.

The storage 33 is realized by an SSD, a flash memory, or the like. A measurement program 32, which is installed in the measurement device 3 and is used to measure the blood glucose equivalent value, is stored in the storage 33 as a storage medium. The CPU 31 reads out the measurement program 32 from the storage 33, develops the measurement program 32 in the memory 36, and executes the developed measurement program 32.

The touch panel 34 is configured with a liquid crystal display, an organic EL, or the like, and performs various displays related to processing performed by the measurement device 3. The touch panel 34 also has a function as an input device for inputting various information to the measurement device 3.

The sensor 38 includes, for example, an infrared light source and an infrared detector, and detects a signal emitted by glucose in blood by irradiating a user who wears the measurement device 3 with infrared rays. The signal detected by the sensor 38 is analyzed by the CPU 31 that executes the measurement program 32, and thus the blood glucose equivalent value is obtained. Further, the sensor 38 may measure an electrocardiogram. In this case, the electrocardiogram is analyzed by the measurement program 32, and thus the blood glucose equivalent value is obtained.

The measurement program 32 is stored in the test server 6 in a state of being accessible from the outside, and is downloaded and installed in the measurement device 3 in response to a request. Alternatively, the measurement program 32 is downloaded in the test apparatus 1 or the mobile terminal 2, and then is downloaded and installed in the measurement device 3 via short-range wireless communication with the test apparatus 1 or the mobile terminal 2.

In the measurement device 3, the sensor 38 detects a signal emitted by glucose in blood at a predetermined time interval. The CPU 31 obtains the blood glucose equivalent value by analyzing the signal by execution of the measurement program 32. Further, the CPU 31 stores the obtained blood glucose equivalent value in the storage 33 in association with a measurement time. The blood glucose equivalent value associated with the measurement time corresponds to a monitoring result of the blood glucose equivalent value. In a case where the user uses the test apparatus 1, the CPU 31 transmits the monitoring result of the blood glucose equivalent value to the test apparatus 1 via the communication I/F 35 according to an instruction of the user or without waiting for an instruction of the user.

Next, the measurement device 4 will be described. FIG. 5 is a hardware configuration diagram of the measurement device 4. As illustrated in FIG. 5, the measurement device 4 is, for example, a measurement device described in JP2016-520379A, which is attached to a human body and measures, as a blood glucose equivalent value, a glucose concentration in an interstitial fluid under epidermis. The measurement device 4 includes a processor 41, a memory 42 as a transitory storage area, a communication I/F 43 for short-range wireless communication, and a sensor 44. A needle-shaped filament 4A that is inserted under the epidermis is connected to the sensor 44. The processor 41, the memory 42, the communication I/F 43, and the sensor 44 are configured with an application specific integrated circuit (ASIC) 45 for measuring glucose in the interstitial fluid.

In the measurement device 4, the sensor 44 detects a signal indicating a glucose concentration in the interstitial fluid under the epidermis at a predetermined time interval or according to a measurement instruction by the user. The processor 41 obtains a glucose concentration by analyzing the signal. Further, the processor 41 stores the obtained glucose concentration in the memory 42 in association with a measurement time. The blood glucose equivalent value associated with the measurement time corresponds to a monitoring result of the blood glucose equivalent value. In a case where the user uses the test apparatus 1, the processor 41 transmits the monitoring result of the blood glucose equivalent value to the test apparatus 1 via the communication I/F 43 according to an instruction of the user or without waiting for an instruction of the user.

Next, a functional configuration of the test apparatus according to the present embodiment will be described. FIG. 6 is a diagram illustrating a functional configuration of the test apparatus according to the present embodiment. As illustrated in FIG. 6, the test apparatus 1 includes an acquisition unit 51, a determination unit 52, a decision unit 53, and a presentation unit 54. The CPU 11 functions as the acquisition unit 51, the determination unit 52, the decision unit 53, and the presentation unit 54 by executing the test program 12. In the following description, it is assumed that the user possesses the measurement device 3 and the test apparatus 1 directly acquires the monitoring result of the blood glucose equivalent value from the measurement device 3.

The acquisition unit 51 acquires the monitoring result of the blood glucose equivalent value transmitted from the communication I/F 35 of the measurement device 3 by receiving the monitoring result of the blood glucose equivalent value by the communication I/F 15. In the present embodiment, for example, the monitoring result of the blood glucose equivalent value measured in the last 24 hours is acquired.

The determination unit 52 determines whether or not there is an abnormal tendency in the blood glucose equivalent value based on the monitoring result of the blood glucose equivalent value acquired by the acquisition unit 51. Specifically, the determination unit 52 determines whether or not the blood glucose equivalent value has a tendency of a postprandial hyperglycemic spike.

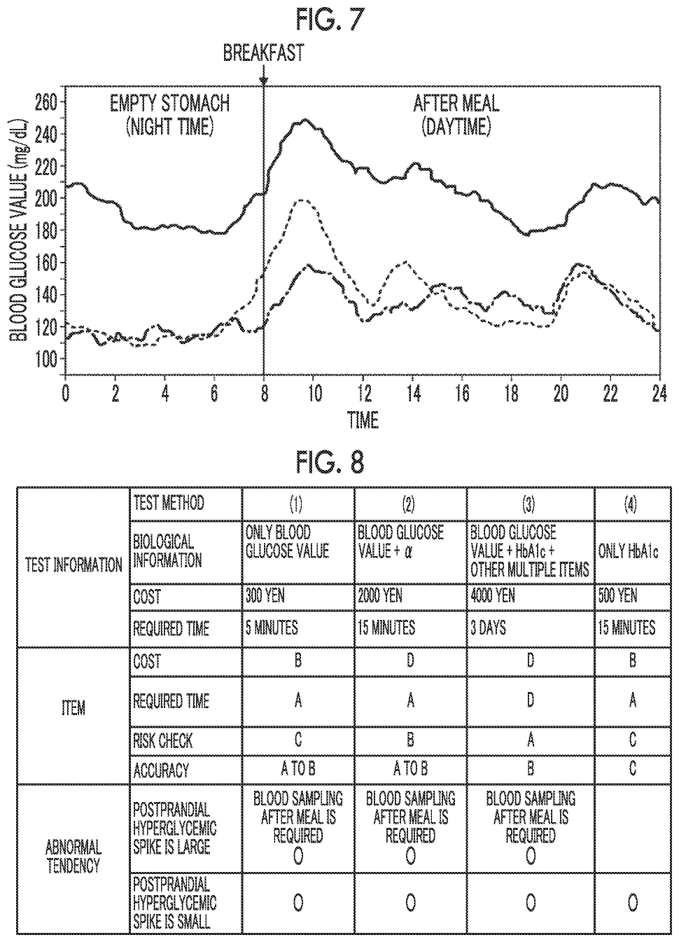

Here, postprandial hyperglycemia will be described. FIG. 7 is a graph illustrating daily variations in blood glucose of a patient with type 2 diabetes. In FIG. 7, a horizontal axis represents a time of one day (24 hours), and a vertical axis represents a blood glucose value (mg/dL). A solid line represents a blood glucose value of a patient whose hemoglobin A1c (HbA1c) is equal to or higher than 9%. A broken line represents a blood glucose value of a patient whose HbA1c is equal to or higher than 7% and lower than 8%. A one-dot line represents a blood glucose value of a patient whose HbA1c is equal to or higher than 6.5% and lower than 7%. Further, FIG. 7 illustrates a variation in the blood glucose value in a case where the patient eats breakfast at around 8:00, eats lunch at around 12:00, and eats dinner at around 19:00. HbA1c is glycated hemoglobin in which hemoglobin as an erythrocyte component in blood is bonded with glucose. HbA1c represents a variation in the blood glucose for 1 to 2 months.

As illustrated in FIG. 7, the blood glucose value increases after meal regardless of a value of HbA1c. On the other hand, as HbA1c is higher, the postprandial hyperglycemic spike indicating that the blood glucose value greatly increases is particularly remarkable after breakfast. In order to make the postprandial hyperglycemic spike after breakfast easy to understand, in FIG. 7, a line is marked at a position indicating 8:00.

In addition, the user transmits a latest meal time from the measurement device 3 to the test apparatus 1. Alternatively, the user may input a meal time via the touch panel 14 of the test apparatus 1. Alternatively, an image of a meal may be captured by the camera 28 of the mobile terminal 2, and the captured meal image may be transmitted to the test apparatus 1. In this case, information on a capturing date and time included in header information of the captured meal image may be used as a meal time.

In the monitoring result of the blood glucose equivalent value transmitted from the measurement device 3, in a case where there is a situation in which a temporal variation in the blood glucose equivalent value after meal is equal to or larger than a predetermined threshold value Th1, the determination unit 52 determines that there is an abnormal tendency in the blood glucose equivalent value. In the present embodiment, the temporal variation is a variation of the blood glucose value per hour.

In the present embodiment, the determination unit 52 further determines whether or not there is a situation in which the variation in the blood glucose equivalent value is equal to or larger than a threshold value Th2. In a case where a determination result is YES, the determination unit 52 determines that there is a large postprandial hyperglycemic spike. Here, Th2>Th1. In a case where a determination result is NO, the determination unit 52 determines that there is a small postprandial hyperglycemic spike.

In a case where it is determined that there is no abnormal tendency in the blood glucose equivalent value, the determination unit 52 notifies the user of a fact that there is no abnormal tendency in the blood glucose equivalent value. The notification will be described later.

In a case where the determination unit 52 determines that there is an abnormal tendency in the blood glucose equivalent value, the decision unit 53 decides a test candidate to be recommended for the user. In addition, the decision unit 53 decides a test candidate according to a degree of the abnormal tendency.

FIG. 8 is a table illustrating test methods for biological information associated with the blood glucose equivalent value. In FIG. 8, for 4-type test methods (1) to (4), a test method, biological information to be measured, a cost, and a required time are illustrated as test information. The test methods (1) to (4) are all test methods including blood sampling of the user.

The test method (1) is a test method for measuring only the blood glucose value. The test method (2) is a test method in which some tests are performed in addition to the blood glucose value. In FIG. 8, some test methods are indicated by “+α”. The test method (3) is a test method for testing the blood glucose value, HbA1c, and multiple items other than the blood glucose value and HbA1c. The test method (4) is a test method for measuring only HbA1c. In FIG. 8, multiple items other than the blood glucose value and HbA1c are indicated by “+other multiple items”. The test methods (1), (2), and (4) can be executed by the analysis apparatus 18 of the test apparatus 1, and in that case, a test result can be acquired in a relatively short time. The acquired test result is displayed on the touch panel 14. The test method (3) is a test in which a sample is delivered to a test center as an external test agency.

The blood glucose value, HbA1c, and other biological information, which are obtained by the test methods (1) to (4), are examples of second biological information.

FIG. 8 illustrates various items serving as indexes in a case where the user selects the presented test candidate. Specifically, various items include an item for a cost required for a test (cost), an item for a time required to obtain a test result (required time), an item for whether to include a risk check for various diseases (risk check), and an item for a test accuracy (accuracy). In a column of each item, a degree of preference is indicated for each test method. The degree of preference is represented by symbols A, B, C, and D in order of preference. A is very preferable, B is preferable, C is neither, and D is not preferable.

In the cost, as the cost is cheaper, the degree of preference is larger. In the required time, as the required time is shorter, the degree of preference is larger. In the risk check for various diseases, the degree of preference is larger in a case where there are many test items and tests for other diseases other than diabetes are included. In the accuracy, as a detection accuracy for postprandial hyperglycemia is higher, the degree of preference is larger.

In addition to the cost, the required time, the risk check, and the accuracy, the items may include a sense of stability in a case where a sample is collected, a degree of restraint in a test, invasiveness or non-invasiveness. In the sense of stability, as a user's anxiety about collecting a sample (for example, blood sampling) is lower, the sense of stability is higher. For example, in a case where the test method includes blood sampling at hospital, blood sampling by a medical staff provides a high sense of stability to the user. In the degree of restraint, the degree of preference varies depending on the presence or absence of going-out and a waiting time. As going-out is less and a waiting time is shorter, the degree of preference is larger. In the invasiveness, the degree of preference is larger in a case where a sample can be obtained without pain. For example, in a case where the test method includes a glucose tolerance test, it is necessary to perform blood sampling three times after glucose tolerance. Thus, the degree of preference for the invasiveness is bad.

Further, FIG. 8 illustrates various abnormal tendencies of the blood glucose equivalent value. Specifically, an abnormal tendency in which a postprandial hyperglycemic spike is large and an abnormal tendency in which a postprandial hyperglycemic spike is small are illustrated.

Further, in FIG. 8, for each abnormal tendency, a column of the test method to be a test candidate is marked with O. That is, in a case where a postprandial hyperglycemic spike is large, the test methods (1) to (3) are marked with O. In the test methods (1) to (3), blood sampling after meal is required for determining a postprandial hyperglycemic spike. In a case where a postprandial hyperglycemic spike is small, all the test methods (1) to (4) are marked with O.

In the present embodiment, the table illustrated in FIG. 8 is stored, as a table, in the storage 13. The decision unit 53 decides a test candidate according to the abnormal tendency of the blood glucose equivalent value determined by the determination unit 52 by referring to the table stored in the storage 13. Specifically, in a case where the abnormal tendency determined by the determination unit 52 corresponds to the abnormal tendency in which a postprandial hyperglycemic spike is large, the decision unit 53 decides the test methods (1) to (3) as test candidates by referring to the table. In a case where the abnormal tendency determined by the determination unit 52 corresponds to the abnormal tendency in which a postprandial hyperglycemic spike is small, the decision unit 53 decides the test methods (1) to (4) as test candidates. In any of the test methods (1) to (4), blood sampling may be performed by the test apparatus 1, and a sample obtained by blood sampling may be delivered to the test center for a test. On the other hand, in the test method (3), for a test, it is necessary to deliver a sample to the test center as an external test agency.

The presentation unit 54 presents, to the user, a test candidate decided by the decision unit 53. In the present embodiment, the presentation unit 54 presents, to the user, a test candidate by displaying the test candidate decided by the decision unit 53 on the touch panel 14.

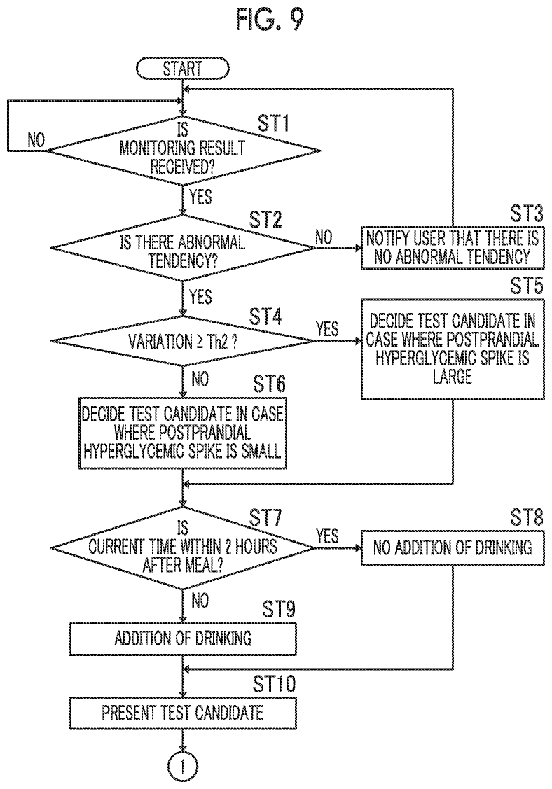

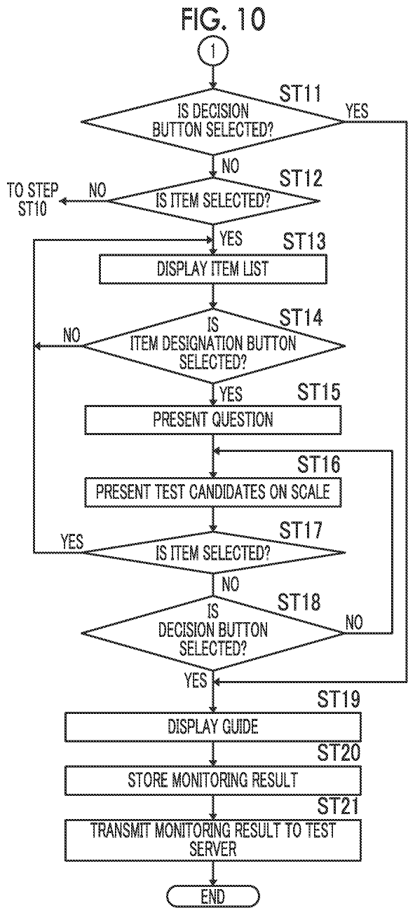

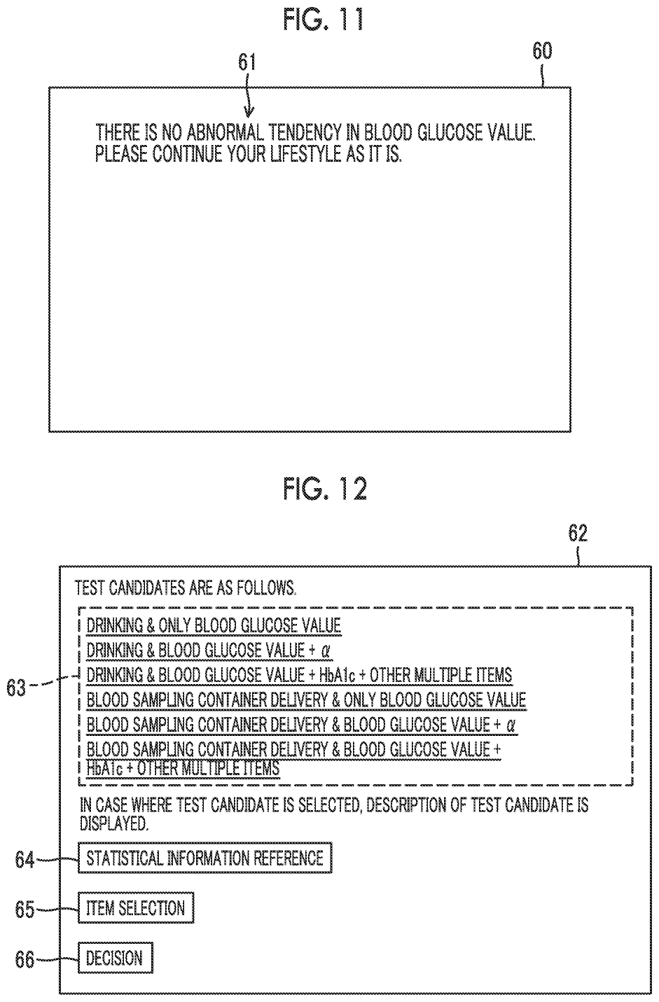

Hereinafter, processing performed in the present embodiment will be described. FIG. 9 and FIG. 10 are flowcharts illustrating processing performed in the present embodiment. In order to determine an abnormal tendency in the blood glucose equivalent value, the acquisition unit 51 monitors whether or not the communication I/F 15 receives the monitoring result of the blood glucose equivalent value transmitted from the measurement device 3 (step ST1). In a case where a monitoring result in step ST1 is YES, the determination unit 52 determines whether or not there is an abnormal tendency in the blood glucose equivalent value based on the monitoring result of the blood glucose equivalent value (step ST2). In a case where a determination result in step ST2 is NO, the determination unit 52 notifies the user that there is no abnormal tendency in the blood glucose equivalent value (step ST3), and the process returns to step ST1. FIG. 11 is a diagram illustrating a notification screen in a case where there is no abnormal tendency in the blood glucose equivalent value. As illustrated in FIG. 11, on the notification screen 60, a notification 61 indicating “There is no abnormal tendency in the blood glucose value. Please continue your lifestyle as it is” is displayed.

In a case where a determination result in step ST2 is YES, the determination unit 52 determines a degree of the abnormal tendency of the blood glucose equivalent value. First, the determination unit 52 determines whether or not there is a situation in which a variation in the blood glucose equivalent value is equal to or larger than a threshold value Th2 based on the monitoring result of the blood glucose equivalent value (step ST4). In a case where a determination result in step ST4 is YES, the decision unit 53 decides, as a test candidate, a test method in a case where a postprandial hyperglycemic spike is large, among the test methods included in the table illustrated in FIG. 8 (step ST5). In a case where a determination result in step ST4 is NO, the decision unit 53 decides, as a test candidate, a test method in a case where a postprandial hyperglycemic spike is small, among the test methods included in the table illustrated in FIG. 8 (step ST6).

After step ST5 and step ST6, the determination unit 52 determines whether or not the current time is within 2 hours after meal based on the meal time (step ST7). In a case where a determination result in step ST7 is YES, the presentation unit 54 does not add drinking of a glucose drink to the test candidate (no addition of drinking, step ST8), and presents the determined test candidate to the user (step ST10). On the other hand, in a case where a determination result in step ST7 is NO, the presentation unit 54 adds drinking of a glucose drink to the test candidate (addition of drinking, step ST9), and presents the determined test candidate to the user (step ST10).

FIG. 12 is a diagram illustrating a test candidate presentation screen. As illustrated in FIG. 12, on the test candidate presentation screen 62, test candidates 63 in a case where a postprandial hyperglycemic spike is large and drinking is included are displayed. That is, for each of “only blood glucose value” of the test method (1), “blood glucose value+α” of the test method (2), and “blood glucose value+HbA1c+other multiple items” of the test method (3), total six test candidates for tests in which drinking is included and a blood sampling container is delivered later are displayed. The test in which drinking is included is displayed as “drinking &”, and the test in which a blood sampling container is delivered later is displayed as “blood sampling container delivery &”. Further, the test candidate presentation screen 62 includes a statistical information reference button 64, an item selection button 65, and a decision button 66. The statistical information reference button 64 is a button for displaying statistical information to be described later. The item selection button 65 is a button for displaying an item list screen including an item serving as an index in a case where the user selects a test candidate. The decision button 66 is a button for deciding a test candidate.

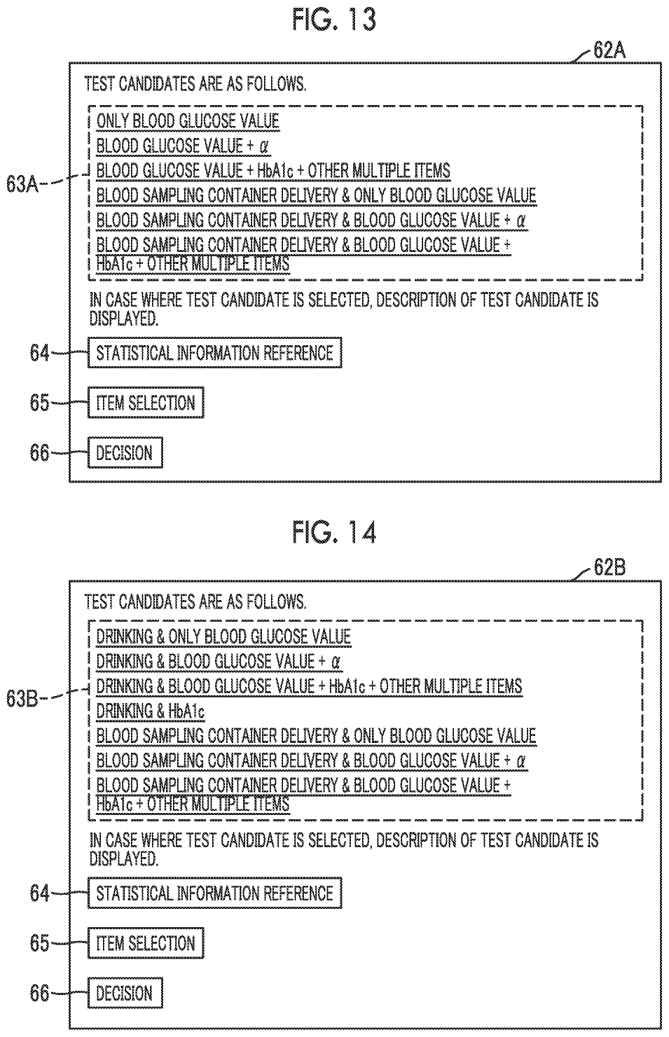

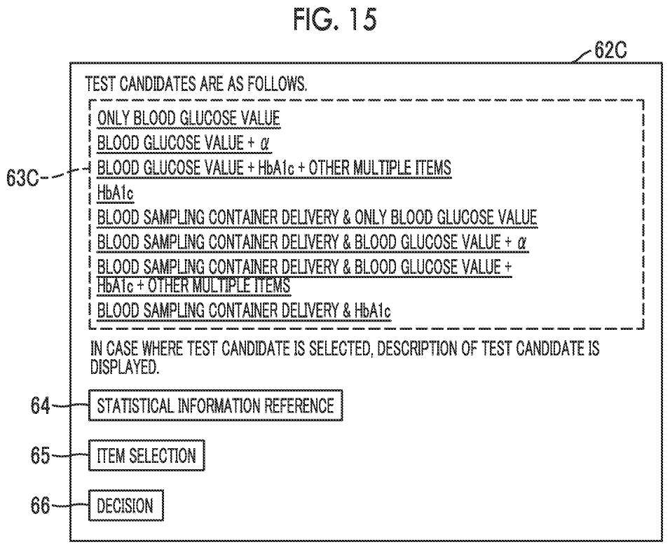

FIG. 13 illustrates a test candidate presentation screen 62A in a case where a postprandial hyperglycemic spike is large and drinking is not included. FIG. 14 illustrates a test candidate presentation screen 62B in a case where a postprandial hyperglycemic spike is small and drinking is included. FIG. 15 illustrates a test candidate presentation screen 62C in a case where a postprandial hyperglycemic spike is small and drinking is not included. On the test candidate presentation screen 62A illustrated in FIG. 13, test candidates 63A in a case where a postprandial hyperglycemic spike is large and drinking is not included are displayed. On the test candidate presentation screen 62B illustrated in FIG. 14, test candidates 63B in a case where a postprandial hyperglycemic spike is small and drinking is included are displayed. On the test candidate presentation screen 62C illustrated in FIG. 15, test candidates 63C in a case where a postprandial hyperglycemic spike is small and drinking is not included are displayed. In a case where a postprandial hyperglycemic spike is small, as the test candidates, “only blood glucose value” of the test method (1), “blood glucose value+α” of the test method (2), “blood glucose value +HbA1c+other multiple items” of the test method (3), and “only HbA1c” of the test method (4) are selected. In the following description, it is assumed that the test candidate presentation screen 62 illustrated in FIG. 12 is displayed.

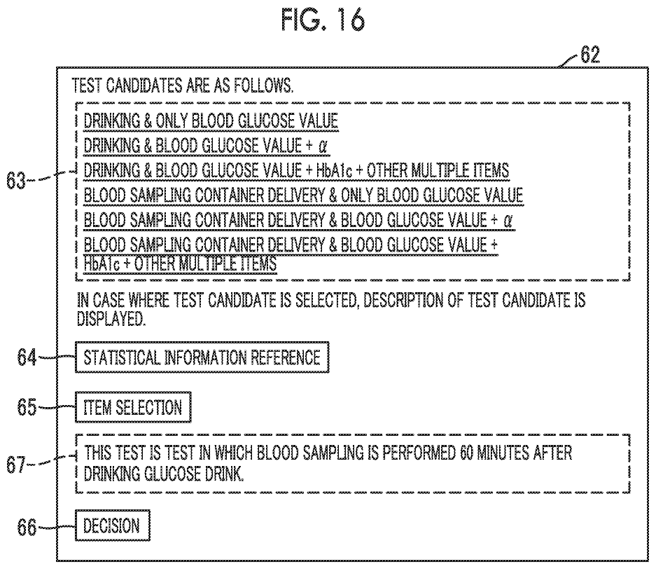

Each test candidate included in the test candidates 63 can be selected, and in a case where the user selects a test candidate included in the test candidates, a description of the selected test candidate is displayed. For example, in a case where the user selects “drinking & only blood glucose value” among the six test candidates, as illustrated in FIG. 16, a description 67 indicating “This test is a test in which blood sampling is performed 60 minutes after drinking a glucose drink.” is displayed between the item selection button 65 and the decision button 66. In a case where the user selects the decision button 66 in this state, the selected test candidate is decided as the test method to be performed by the user. Processing after the test method is decided will be described later.

Further, in a case where the user selects the statistical information reference button 64, statistical information of the test candidate which is previously selected is displayed. Here, plural test candidates are selected and presented to the user according to the abnormal tendency of the blood glucose equivalent value of the user, and the user selects one test candidate among the plurality of test candidates and performs the test. The statistical information represents a ratio that each of the plurality of test candidates is previously selected by the user who currently performs a test and/or another user. The statistical information is obtained by counting the test candidates which are previously selected from the test candidates having the same abnormal tendency as the abnormal tendency of the blood glucose equivalent value of the user. For example, in a case where the abnormal tendency of the blood glucose equivalent value of the user is an abnormal tendency in which a postprandial hyperglycemic spike is large, the statistical information for the presented six test candidates is displayed.

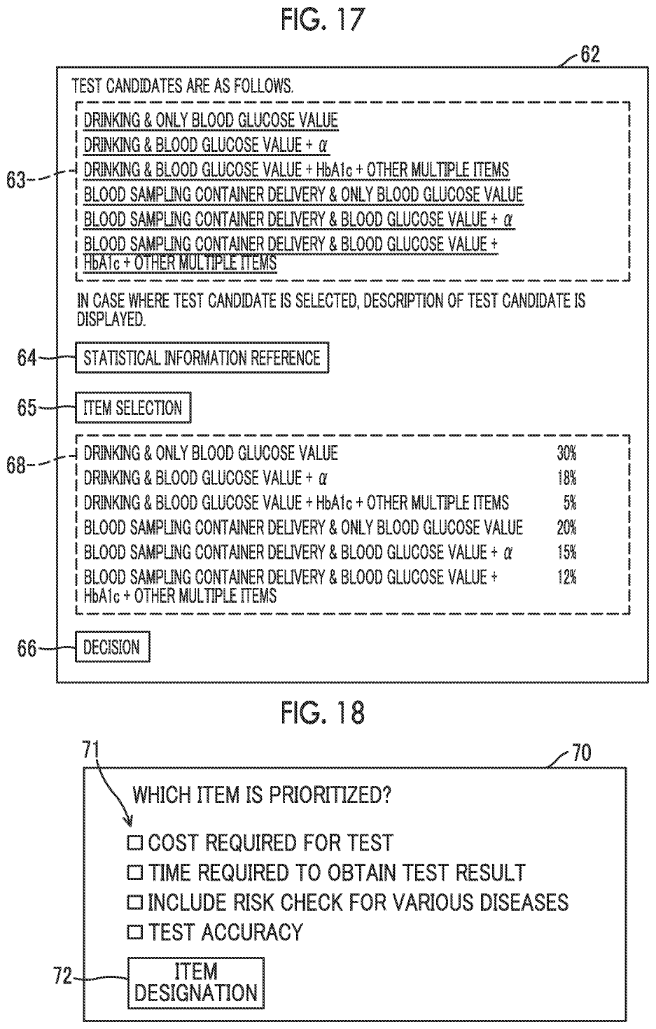

FIG. 17 is a diagram illustrating a test candidate presentation screen on which statistical information of the test candidate is displayed. As illustrated in FIG. 17, statistical information 68 for each of the presented six test candidates is displayed between the item selection button 65 and the decision button 66. As illustrated in FIG. 17, in the statistical information 68, the ratio that each test candidate is previously selected is indicated by a percentage. The statistical information 68 is stored in the test server 6. In a case where the statistical information reference button 64 is selected, the mobile terminal 2 accesses the test server 6, acquires the statistical information 68, and presents the statistical information 68 to the user.

Even in a state where the statistical information 68 is displayed, in a case where the user selects one of the test candidates and selects the decision button 66, the selected test candidate is decided as the test method to be performed by the user.

After the test candidates are presented, the decision unit 53 determines whether or not the decision button 66 is selected (step ST11), and in a case where a determination result in step ST11 is YES, the process proceeds to step ST19 to be described later. In a case where a determination result in step ST11 is NO, the decision unit 53 determines whether or not the item selection button 65 is selected (item selection, step ST12). In a case where a determination result in step ST12 is NO, the process returns to step ST10. In a case where a determination result in step ST12 is YES, the presentation unit 54 displays an item list (step ST13).

FIG. 18 is a diagram illustrating a list screen for displaying an item list. As illustrated in FIG. 18, a text “which item is prioritized?” is displayed on the item list screen 70. Further, on the item list screen 70, as an item serving as an index in a case where the user selects a test candidate, a cost required for the test, a time required to obtain a test result, a risk check for various diseases, and a test accuracy are displayed. Further, a check box 71 is added to each item. The user designates an item by checking the check box of a certain item that the user wants to prioritize in a case of selecting the test information, and selects an item designation button 72. For this reason, the decision unit 53 determines whether or not the item designation button 72 is selected (step ST14). In a case where a determination result in step ST14 is NO, the process returns to step ST13. In a case where a determination result in step ST14 is YES, the decision unit 53 presents, to the user, a question for determining a display position of the test candidate according to the designated item on a scale to be described (step ST15).

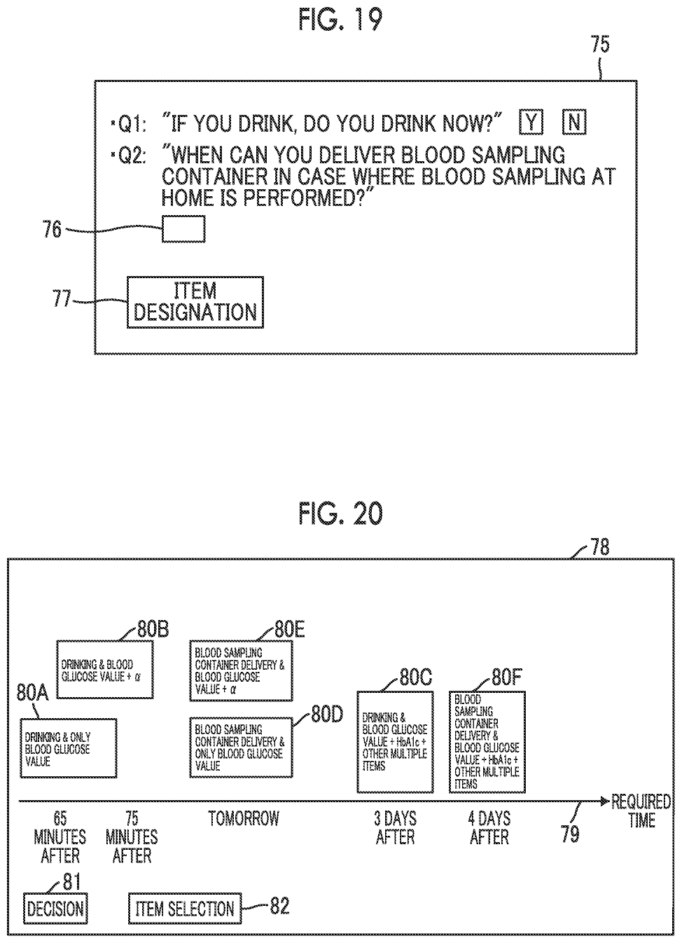

FIG. 19 is a diagram illustrating a question presentation screen. For the sake of explanation, it is assumed that the user selects “a required time to obtain a test result” as an item serving as an index in a case of selecting a test candidate. As illustrated in FIG. 19, two questions Q1 and Q2 are displayed on the question presentation screen 75. The question Q1 is “If you drink, do you drink now?”, and the question Q2 is “When can you deliver a blood sampling container in a case where blood sampling at home is performed?”. The user answers YES or NO to the question Q1. For the question Q2, the user inputs, in an input box 76, a date and time when a blood sampling container will be delivered. Here, it is assumed that the user answers YES to the question Q1 and inputs tomorrow's date to the question Q2.

In a case where the user inputs an answer and then selects an answer completion button 77, the decision unit 53 decides a position for displaying the test candidate on a scale according to the designated item. The presentation unit 54 presents the test candidates to the user in a display form in which icons representing the test candidates are displayed, at the decided display positions on the scale (presentation of the test candidates on scale, step ST16). Here, in the present embodiment, “a required time to obtain a test result” is selected as an item serving as an index in a case where the user selects a test candidate. Thus, the decision unit 53 obtains a required time to obtain a test result according to the answer to the question, and decides a display position of the test candidate on a scale representing the required time. The presentation unit 54 presents the test candidates to the user in a display form in which test candidate icons are displayed at the decided display positions on the scale.

FIG. 20 is a diagram illustrating a presentation screen for presenting the test candidates to the user in a display form in which test candidate icons are displayed on the scale. As illustrated in FIG. 20, on the presentation screen 78, a scale 79 representing the required time is displayed, and six test candidate icons 80A to 80F are displayed so as to be arranged along the scale 79 according to the required time. As illustrated in FIG. 20, the display position of the test candidate icon 80A corresponding to “drinking & only blood glucose value” on the scale 79 is “65 minutes after”. This is because an answer to the question Q1 is to drink now, blood sampling is performed 60 minutes after drinking, and a result is obtained 5 minutes after a start of the test. In addition, the display position of the test candidate icon 80B corresponding to “drinking & blood glucose value+α” on the scale 79 is “75 minutes after”. This is because an answer to the question Q1 is to drink now, blood sampling is performed 60 minutes after drinking, and a result is obtained 15 minutes after a start of the test. Further, the display position of the test candidate icon 80C corresponding to “drinking & blood glucose value+HbA1c+other multiple items” on the scale 79 is “3 days after”. This is because an answer to the question Q1 is to drink now but it will take 3 days until all test results are obtained.

The display position of the test candidate icon 80D corresponding to “blood sampling container delivery & only blood glucose value” and the display position of the test candidate icon 80E corresponding to “blood sampling container delivery & blood glucose value+α” on the scale 79 are “tomorrow”. This is because an answer to the question Q2 is to deliver a blood sampling container tomorrow. The display position of the test candidate icon 80F corresponding to “blood sampling container delivery & blood glucose value+HbA1c+other multiple items” on the scale 79 is “4 days after”. This is because an answer to the question Q2 is to deliver a blood sampling container tomorrow and it will take another 3 days until all test results are obtained.

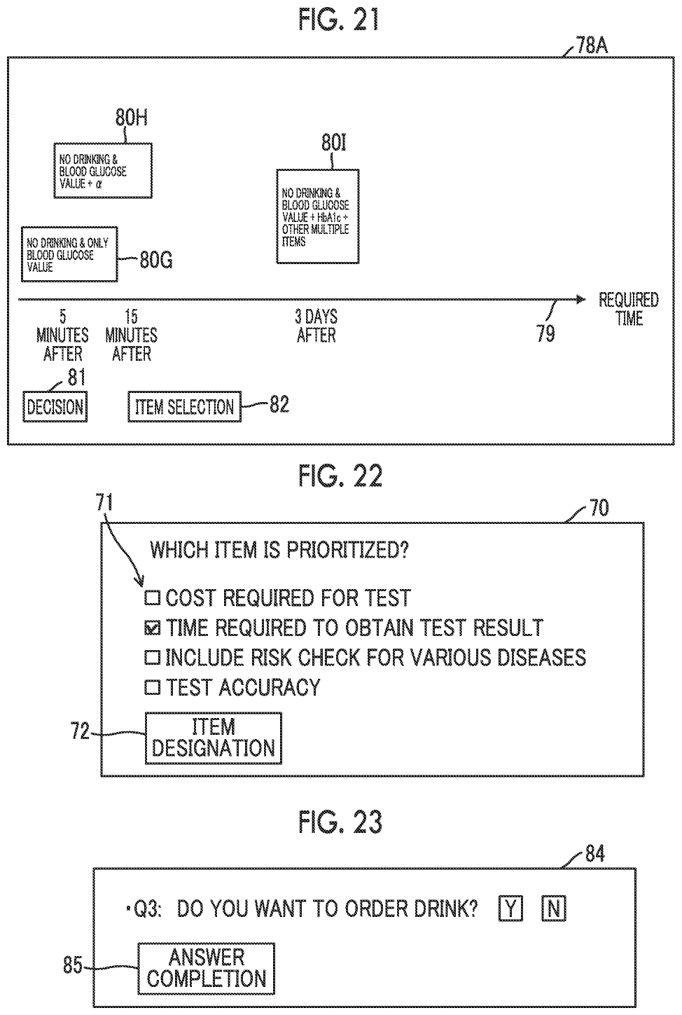

FIG. 21 illustrates a test candidate presentation screen in a case where the user is in a state of being within 2 hours after meal, the presentation screen 62A illustrated in FIG. 13 is displayed, there is a question as to whether to perform blood sampling immediately, and the user answers YES to the question. In the test candidates 63A on the presentation screen 62A illustrated in FIG. 13, no drinking is not indicated. On the other hand, in the icons illustrated in FIG. 21, no drinking is indicated. On the presentation screen 78A illustrated in FIG. 21, the display position of the test candidate icon 80G corresponding to “no drinking & only blood glucose value” on the scale 79 is “5 minutes after”. This is because a result is obtained 5 minutes after the start of the test. In addition, the display position of the test candidate icon 80H corresponding to “no drinking & blood glucose value+α” on the scale 79 is “15 minutes after”. This is because a result is obtained 15 minutes after the start of the test. Further, the display position of the test candidate icon 801 corresponding to “no drinking & blood glucose value+HbA1c+other multiple items” on the scale 79 is “3 days after”. This is because it will take 3 days until all test results are obtained.

The user can recognize the required time for the presented test candidate at a glance based on the presentation screen 78. Thereby, it is possible to easily compare the test candidates according to the item that the user prioritizes.

The user can decide a test method desired by the user by selecting a test candidate icon and selecting a decision button 81 on the presentation screen 78. Further, the user can additionally designate an item serving as an index in a case of selecting a test candidate, by selecting an item selection button 82. Thus, after step ST16, the decision unit 53 determines whether or not the item selection button 82 is selected (step ST17). In a case where a determination result in step ST17 is YES, the process returns to step ST13, and processing of step ST13 to step ST17 is repeated. In a case where a determination result in step ST17 is NO, the decision unit 53 determines whether or not the decision button 81 is selected (step ST18). In a case where a determination result in step ST18 is YES, the process proceeds to step ST19 to be described later. In a case where a determination result in step ST18 is NO, the process returns to step ST16.

FIG. 22 is a diagram illustrating an item list screen displayed twice in a case where a determination result in step ST17 is YES. As illustrated in FIG. 22, among the check boxes 71 displayed on the item list screen 70, a check box of “a required time to obtain a test result” is already checked. The user can further select an item on the item list screen 70. Here, it is assumed that the user further selects “a cost required for the test”. The decision unit 53 presents, to the user, a question for deciding the display position of the test candidate on the scale according to “a cost required for the test”.

FIG. 23 is a diagram illustrating a question presentation screen related to “a cost required for the test”. As illustrated in FIG. 23, one question Q3 is presented on the question presentation screen 84. The question Q3 is “Do you want to order a drink?” The user answers YES or NO to the question. Here, it is assumed that the user answers YES to the question. After the answering, in a case where the user selects the answer completion button 85, the presentation unit 54 updates a display form of the icon representing the test candidate. That is, the decision unit 53 decides a display position of the test candidate on an additional scale according to the additionally-designated item, and the presentation unit 54 presents, to the user, the test candidates at the decided display positions on the presented scale and the additional scale, in a display form in which icons representing the test candidates are displayed.

Specifically, the decision unit 53 obtains a cost required for the test according to the answer to the question, and decides a display position of the test candidate on the scale representing the cost required for the test. The presentation unit 54 presents the test candidates to the user by displaying the scale of the required time and the scale of the cost required for the test such that the two scales are perpendicular to each other and displaying the test candidate icons at the positions on the two scales perpendicular to each other.

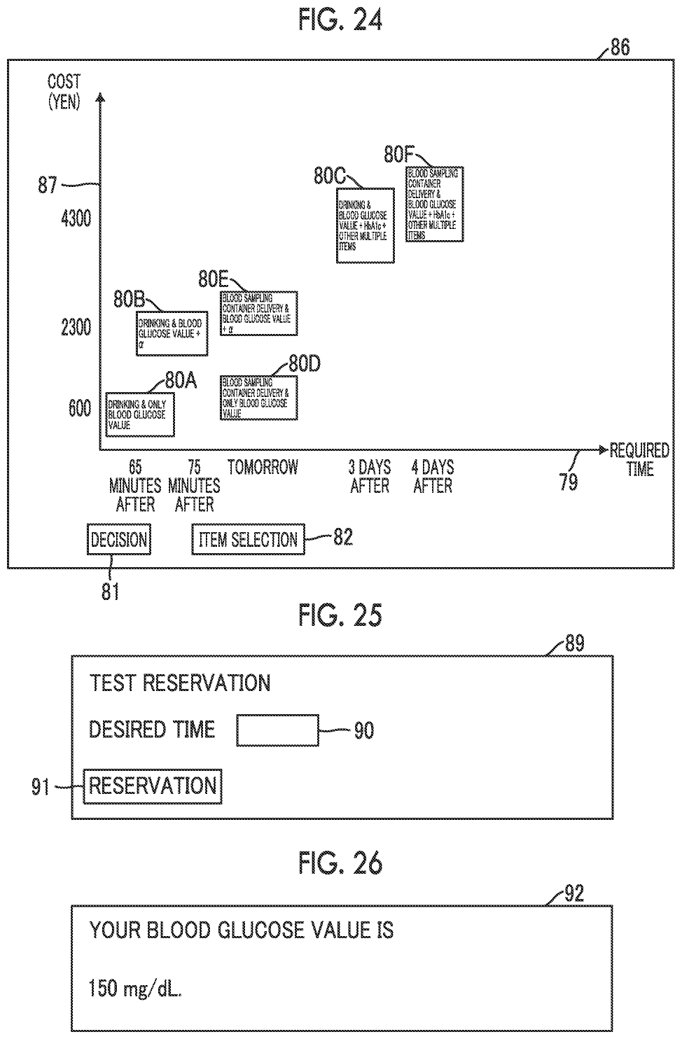

FIG. 24 is a diagram illustrating a presentation screen for presenting the test candidates to the user in a display form in which the test candidate icons are displayed on the scale in a case where two items are selected. As illustrated in FIG. 24, on the presentation screen 86, a scale 79 representing the required time and a scale 87 representing the cost are displayed so as to be perpendicular to each other, and six test candidate icons 80A to 80F are displayed so as to be arranged in a two-dimensional shape according to the required time and the cost. As illustrated in FIG. 24, the display position of the test candidate icon 80A corresponding to “drinking & only blood glucose value” on the scale 79 is “65 minutes after”, and the display position of the test candidate icon 80A on the scale 87 is “600 yen”. The cost is obtained by adding a charge of the glucose drink to the test cost for only a blood glucose value.

Further, the display position of the test candidate icon 80B corresponding to “drinking & blood glucose value+α” on the scale 79 is “75 minutes after”, and the display position of the test candidate icon 80B on the scale 87 is “2300 yen”. The cost is obtained by adding a charge of the glucose drink to the test cost for a blood glucose value+α. Further, the display position of the test candidate icon 80C corresponding to “drinking & blood glucose value+HbA1c+other multiple items” on the scale 79 is “3 days after”, and the display position of the test candidate icon 80C on the scale 87 is “4300 yen”. The cost is obtained by adding a charge of the glucose drink to the test cost for a blood glucose value+HbA1c+other multiple items.

The display position of the test candidate icon 80D corresponding to “blood sampling container delivery & only blood glucose value” and the display position of the test candidate icon 80E corresponding to “blood sampling container delivery & blood glucose value+α” on the scale 79 are “tomorrow”, and the display positions of the test candidate icons 80D and 80E on the scale 87 are slightly higher than the display positions of the test candidate icons 80A and 80B. This is because a transportation cost (for example, 300 yen) when the blood sampling container is delivered to the test apparatus 1 again is included into the costs for “drinking & only blood glucose value” and “drinking & blood glucose value+α”. Further, the display position of the test candidate icon 80F corresponding to “blood sampling container delivery & blood glucose value+HbA1c+other multiple items” on the scale 79 is “4 days after”, and the display position of the test candidate icon 80F on the scale 87 is slightly higher than the display position of the test candidate icon 80C. This is because a transportation cost when the blood sampling container is delivered to the test apparatus 1 again is included into the cost for “drinking & blood glucose value+HbA1c+other multiple items”.

The user can recognize the required time and the cost for the presented test candidate at a glance based on the presentation screen 86. Thereby, it is possible to easily compare the test candidates in consideration of both the required time and the cost.

The user can decide the test method desired by the user by selecting the test candidate icon and selecting the decision button 81 on the presentation screen 86. Further, the user can additionally designate an item serving as an index in a case of selecting a test candidate, by further selecting the item selection button 82. Thereby, it is possible to compare the test candidates until the user is satisfied by further adding an item.

In a case where a determination result in step ST11 is YES, or in a case where a determination result in step ST18 is YES, the presentation unit 54 displays a guide on the test method selected by the user on the touch panel 14 (step ST19). For example, in a case where the user selects “drinking & only blood glucose value” on the presentation screen 62 or the like, blood sampling is required after 60 minutes. Thus, the presentation unit 54 displays a test reservation guide screen. FIG. 25 is a diagram illustrating a test reservation guide screen. On the reservation guide screen 89 illustrated in FIG. 25, the user inputs a desired reservation time in an input box 90. Thereafter, in a case where a reservation button 91 is selected, the reservation is completed. The test apparatus 1 is controlled from several minutes before a time when reservation is performed by the user so as not to acquire the monitoring result of a blood glucose equivalent value of another user and not to perform a test for another user.

After step ST19, the decision unit 53 stores the acquired monitoring result of the blood glucose equivalent value of the user in the storage 13 in association with the acquisition date and time and the abnormal tendency (step ST20). Further, the decision unit 53 associates the acquired monitoring result of the blood glucose equivalent value of the user with the acquisition date and time, the abnormal tendency, and the selected test candidate, processes the information such that the user is not specified, and transmits the processed information to the test server 6 (step ST21). Then, the process is completed. In a case where the image of the meal is acquired, the image is also transmitted to the test server 6. In the test server 6, the acquired information is stored.

The user goes to a location of the test apparatus 1 at a reserved time, and performs blood sampling by using the analysis apparatus 18 or loads, into the analysis apparatus 18, the blood sampling container in which the blood collected by using the test kit is contained. Thereby, the analysis apparatus 18 obtains an analysis result by analyzing the blood according to the test method selected by the user. The analysis result is displayed on the touch panel 14. FIG. 26 is a diagram illustrating an analysis result display screen. As illustrated in FIG. 26, on the analysis result display screen 92, as an example, “150 mg/dL”, which is the analysis result of the blood glucose value, is displayed.

As described above, in the present embodiment, in a case where there is an abnormal tendency in the monitoring result of the blood glucose equivalent value of the user, the test candidates for acquiring biological information associated with the blood glucose equivalent value are presented to the user, and a guide according to the test candidate selected by the user is presented to the user. Therefore, the user can perform a test according to the monitoring result of the blood glucose equivalent value by using the test apparatus according to the present embodiment. In addition, the user will not perform a test that does not match with the abnormal tendency of his/her blood glucose value. Therefore, it is possible to provide personalized medical care suitable for the user.

In addition, by presenting the test candidates that can acquire biological information associated with the blood glucose equivalent value with higher accuracy than accuracy of the measurement device 3 or 4, the user can select a test by which his/her blood glucose status can be accurately recognized. Therefore, in a case where the selected test is performed, the user can more accurately recognize his/her blood glucose status.

Further, by receiving designation of at least one item serving as an index in a case where the user selects a test candidate and presenting test candidates in a display form according to the designated item, the test candidates can be presented in a display form which reflects the item that the user wants to prioritize in a case of selecting a test candidate. Therefore, the user can easily select the test candidate.

In addition, by presenting, to the user, a question for determining the display position of the test candidate on the scale according to the designated item, deciding the display position of the test candidate on the scale according to the answer to the question, and presenting, to the user, the test candidate in a display form in which an icon representing the test candidate is displayed at the decided display position on the scale, the test candidate can be presented to the user in a display form which reflects an item serving as an index in a case where the user selects the test candidate. Therefore, the user can select the test candidate in consideration of the item that he/she prioritizes.

Further, by receiving additional designation of at least one item serving as an index in a case where the user selects a test candidate and presenting, to the user, the test candidates by updating the display form of the icons representing the test candidates according to the additionally-designated item, the test candidates can be presented in a display form in which the plurality of items serving as indexes in a case where the user selects a test candidate are considered. Therefore, the user can easily select the test candidate.

Further, by presenting the statistical information of the test candidates which are previously selected to the user, the user can more easily select the test method suitable for himself/herself.

Further, in a case where a blood glucose equivalent value, an abnormal tendency, a selected test candidate, an image of a meal, and the like are stored in the test server 6, the stored information may be used as big data. The stored big data may be used for learning AI that provides information related to blood glucose values, or may be used as statistical information.

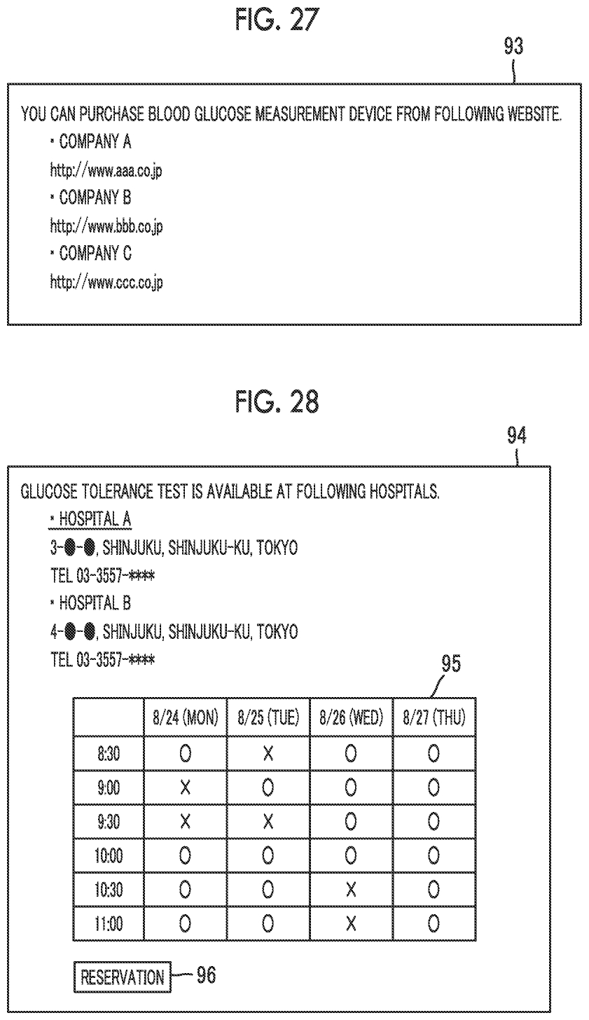

In the first embodiment, a test method that cannot be performed by the test apparatus 1 may be presented as a test candidate. For example, a test in which the user performs self blood sampling on a regular basis or a test method in which blood sampling is performed at a hospital may be presented as a test candidate. In a state where such a test method is presented as a test candidate, for example, in a case where the user selects a test method including self blood sampling at home and blood glucose measurement because he/she wants to check a blood glucose value on a regular basis in the future, the presentation unit 54 provides, to the user, a guide on a website that sells a blood glucose measurement device (self monitoring of blood glucose, SMBG). FIG. 27 is a diagram illustrating a guide screen on a website that sells a blood glucose measurement device. As illustrated in FIG. 27, on the guide screen 93, plural websites on which the user can purchase a blood glucose measurement device are displayed. The user can purchase a blood glucose measurement device by accessing the displayed web site.

On the other hand, in a state where the test methods to be performed at hospital are presented as test candidates, in a case where the user intends to properly check a current state of his/her blood glucose value and selects the glucose tolerance test at hospital, the presentation unit 54 provides, to the user, a guide on a hospital which is located near the user's home or workplace and at which the glucose tolerance test can be performed. FIG. 28 is a diagram illustrating a guide screen for guiding, to the user, a hospital at which the glucose tolerance test can be performed. As illustrated in FIG. 28, on the guide screen 94, a hospital which is located near the user's workplace and at which the glucose tolerance test can be performed is displayed. In addition, a presented hospital name can be selected, and in a case where the hospital name is selected, a list 95 of dates and times for reservation of the glucose tolerance test is displayed. FIG. 28 illustrates a state where a hospital A is selected and a list of dates and times for reservation of the hospital A is displayed. The user can make a reservation for the glucose tolerance test at the selected hospital by selecting the date and time on which “O” is marked and selecting a reservation button 96.

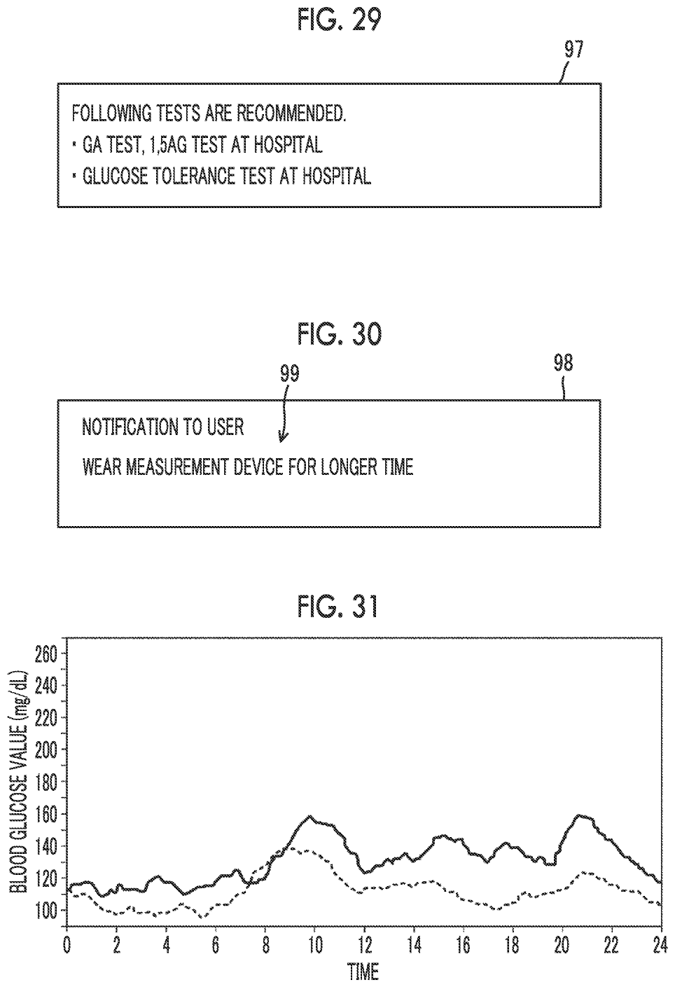

In the first embodiment, in a case where the user selects a test from the test candidates, a guide is provided and the process is completed. On the other hand, in a case where the user selects a test, the test candidates may be suggested as step-up processing. For example, as illustrated in FIG. 12, in a state where the postprandial hyperglycemic spike is large and six test candidates are presented, in a case where the user selects any one of the test candidates, as a next test candidate, tests at hospital may be guided. FIG. 29 is a diagram illustrating a guide screen for the next test. As illustrated in FIG. 29, on the guide screen 97, as a next step test, two test methods including a GA test and a 1,5AG test at hospital and a glucose tolerance test at hospital are presented. By referring to the guide screen 97, the user can recognize that it is preferable to perform a GA test and a 1,5AG test at hospital or a glucose tolerance test at hospital, as a next test.

It is noted that a GA test is a glycoalbumin test and a 1,5AG test is a 1,5-anhydro-D-glucitol test. The glucoalbumin is a combination of albumin, which is a kind of protein in serum, and glucose, and is biological information by which a state of the blood glucose value before one week to two weeks can be recognized. 1,5AG is sugar in blood, and is the second highest amount of sugar after glucose. By measuring 1,5AG as biological information, a state of the blood glucose value for previous several days can be recognized. The glucose tolerance test is a test for measuring a blood glucose value by performing blood sampling three times after glucose tolerance.

Further, in the first embodiment, for example, in a case where a measured value of the blood glucose equivalent value is insufficient, whether or not there is an abnormal tendency in the blood glucose value may not be determined. For example, in a case where the user wears the measurement device 3 for a short time, the blood glucose equivalent value may not be monitored enough to determine the postprandial hyperglycemic spike. In such a case, the determination unit 52 cannot determine whether or not there is an abnormal tendency. Thus, in a case where the determination unit 52 cannot determine whether or not there is an abnormal tendency, preferably, a notification urging the user to wear the measurement device 3 for a longer time is displayed. FIG. 30 is a diagram illustrating a notification screen. As illustrated in FIG. 30, on a notification screen 98, a notification 99 indicating “Please wear the measurement device for a longer time.” is displayed. Based on the notification screen 98, the user can take an action to wear the measurement device 3 for a longer time. Thereby, it is possible to determine whether or not there is an abnormal tendency in the blood glucose equivalent value after the action.

Further, in the first embodiment, the user may capture an image of meal content by using the camera 28 of the mobile terminal 2, and transmit the image of meal content and the monitoring result of the blood glucose equivalent value to the test apparatus 1. In this case, in the test apparatus 1, in a case where the current time is within 2 hours after meal, the meal content may be determined based on the image of the meal content, and the test candidate may be determined according to the meal content. For example, in a case where the abnormal tendency determined by the determination unit 52 corresponds to the abnormal tendency in which a postprandial hyperglycemic spike is small, the decision unit 53 determines meal content. In a case where the meal content is low in glucose, it is considered that a postprandial hyperglycemic spike is small due to an influence of the meal. Thus, the decision unit 53 may change the abnormal tendency of the blood glucose equivalent value to the abnormal tendency in which a postprandial hyperglycemic spike is large, and decide the test methods (1) to (3) as test candidates.

Further, in the first embodiment, on the item list screen 70, every time one item serving as an index in a case where the user selects a test candidate is selected, the test candidates are presented to the user in a display form using a scale. On the other hand, the present disclosure is not limited thereto. On the item list screen 70, the plurality of items may be selected at once. In this case, the display positions of the test candidates on the scale of each of the plurality of selected items are decided, and thus the test candidates are presented to the user in a display form in which the test candidate icons are displayed at the decided display positions.

Further, in the first embodiment, in a case where the item selection button 65 is selected, the item list screen 70 is displayed such that an item serving as an index in a case where the user selects a test candidate can be selected by the user. On the other hand, the present disclosure is not limited thereto. Before the test apparatus according to the present embodiment determines an abnormal tendency based on the monitoring result of the blood glucose equivalent value, an item serving as an index in a case where the user selects a test candidate may be selected in advance by the user. Alternatively, an item serving as an index in a case where the user selects a test candidate may be transmitted from the measurement device 3 to the test apparatus 1. In this case, instead of the item selection button 65, a button is displayed on the test candidate presentation screen 62. In a case where the button is selected, the test candidates are presented to the user in the display form illustrated in FIG. 21, FIG. 22, or FIG. 24.

In a case where an item serving as an index in a case where the user selects a test candidate is selected in advance by the user, the user may select the item including a risk check for various diseases. In such a case, the decision unit 53 may decide, as a test candidate, a test method including a test for other multiple items. For example, the test methods (2) and (3) illustrated in FIG. 8 may be decided as test candidates.

Further, in the first embodiment, in a case where, on the item list screen 70, an item serving as an index in a case where the user selects a test candidate is selected, a question is presented to the user, and the display positions of the test candidates on the scale are decided according to an answer to the question. On the other hand, the present disclosure is not limited thereto. The display positions of the test candidates on the scale may be decided according to a degree of preference of the selected item without a question. For example, in a case where the user selects the test accuracy, the display positions are decided such that an icon of a test candidate with a higher test accuracy is displayed at a position with a higher test accuracy on the scale representing the test accuracy.

Further, in the first embodiment, in a case where there is no abnormal tendency in the blood glucose equivalent value, as illustrated in FIG. 11, a notification indicating “There is no abnormal tendency in the blood glucose value. Please continue your lifestyle as it is” is displayed. On the other hand, the content of the notification is not limited thereto. In a case where there is no abnormal tendency in the blood glucose equivalent value but there is an increase tendency in the blood glucose equivalent value, preferably, a notification including an advice for warning the user to be careful about lifestyle is displayed.

Further, in the first embodiment, the blood glucose equivalent value is measured by the measurement devices 3 and 4. On the other hand, the present disclosure is not limited thereto. Instead of measuring the blood glucose equivalent value by the measurement devices 3 and 4, a urine glucose value may be measured by performing a urine glucose test. In this case, the measured urine glucose value is an example of the first biological information. In a case of the urine glucose test, since a urine glucose value is obtained by using a urine glucose test apparatus, the urine glucose value may be used as a blood glucose equivalent value. The urine glucose test apparatus is an example of a measurement device. In this case, the urine glucose test apparatus has a communication function with the mobile terminal 2, and the measured urine glucose value is transmitted to the mobile terminal 2. Thereby, the mobile terminal 2 may monitor the blood glucose equivalent value based on the urine glucose value.

On the other hand, in a case where a urine glucose test is performed by using a test paper, a color of the test paper is changed according to urine glucose. In this case, an image of the test paper may be acquired by capturing the test paper by using the camera 28 of the mobile terminal 2, and the mobile terminal 2 may recognize the color of the test paper from the image of the test paper. Thereby, the urine glucose value may be stored, and the stored urine glucose value may be transmitted from the mobile terminal 2 to the test apparatus 1 as a monitoring result of the blood glucose equivalent value. In this case, no measurement device is required, and thus the user does not need to prepare the measurement devices 3 and 4 or a urine glucose test apparatus. Further, in this case, the mobile terminal 2 may determine the abnormal tendency in the blood glucose equivalent value from the acquired urine glucose value.

Further, the blood glucose equivalent value may be monitored by using an ambulatory glucose profile (AGP) instead of the blood glucose equivalent value measured by the measurement devices 3 and 4. AGP is an analysis method that is useful for reading a tendency of a variation in blood glucose, that is, a blood glucose trend, from blood glucose values for several days obtained by continuous measurement or a glucose value in an interstitial fluid (https://dm-net.co.jp/trend/agp/001.php). By using AGP, it becomes easy to recognize a time zone in which hypoglycemia and hyperglycemia are likely to occur during a day and a time zone in which a variation in the blood glucose value is large. Therefore, by monitoring the blood glucose equivalent value by using AGP, it is possible to easily determine an abnormal tendency of the blood glucose equivalent value.

Further, in the first embodiment, the blood glucose equivalent value measured by the measurement devices 3 and 4 is monitored, and thus a comparison result between a monitoring result of the blood glucose equivalent value and a test result obtained by blood sampling of the user may be presented to the user. FIG. 31 is a graph illustrating a comparison result between a monitoring result of the blood glucose equivalent value and a test result obtained by blood sampling of a user. In FIG. 31, a solid line represents a monitoring result of the blood glucose equivalent value, and a broken line represents a test result obtained by blood sampling. As illustrated in FIG. 31, the blood glucose equivalent value measured by the measurement devices 3 and 4 may tend to be higher than the actual blood glucose value. As illustrated in FIG. 31, a comparison result between a monitoring result of the blood glucose equivalent value and a test result obtained by blood sampling of the user is presented to the user. Thereby, the user can determine a deviation between the current blood glucose equivalent value and the actual blood glucose value.

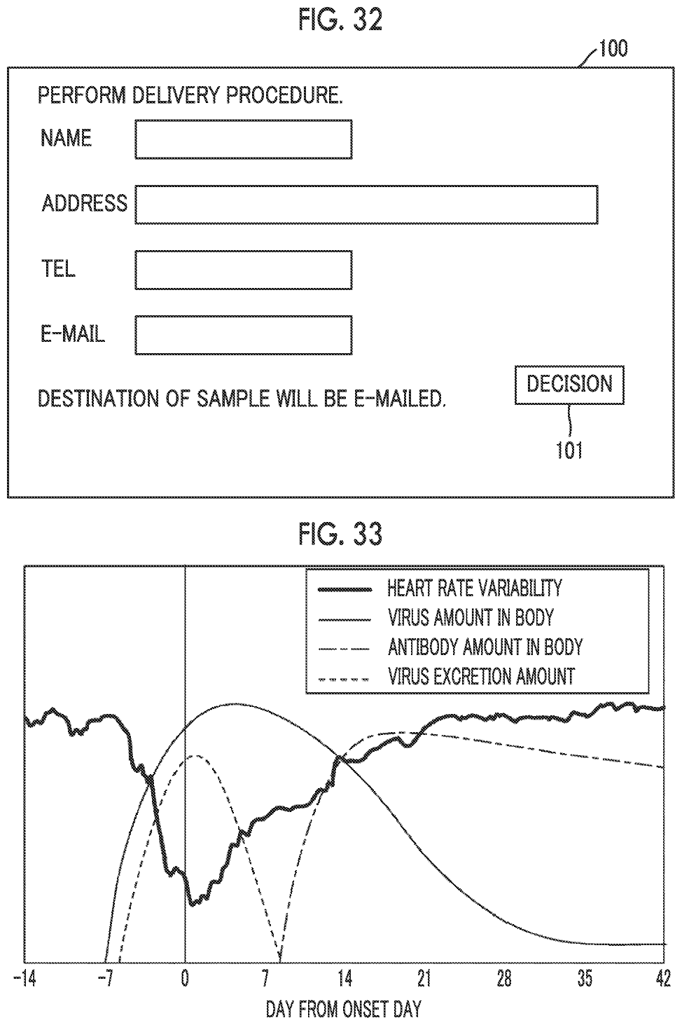

Further, in the first embodiment, in a case where the test method (3) is selected, the blood collected by the user is delivered to the test center. Further, even in a case where the test methods (1), (2), and (4) are selected, the blood collected by the user may be delivered to the test center for a desired test. In this case, the presentation unit 54 of the test apparatus 1 displays a guide screen for performing a delivery procedure on the touch panel 14. FIG. 32 is a diagram illustrating a guide screen for performing a delivery procedure of a test result. As illustrated in FIG. 32, on the guide screen 100, a box for inputting a name, an address, a telephone number, and an e-mail address of a user is displayed. The user inputs necessary items and selects a decision button 101. Thereby, a destination of the sample is e-mailed to the e-mail address of the user. The user delivers the sample to the destination emailed to the his/her e-mail address. Further, a test result will be delivered to the user at a later date. On the other hand, the test result can also be confirmed in the test apparatus 1. In this case, the test result is transmitted from the test center to the test apparatus 1. In a case where the user inputs his/her own ID and the like on the test apparatus 1, the test apparatus 1 can display the test result.

The user performs user registration in order to use the test apparatus 1. In a case where user information for user registration such as the user's name, address, and telephone number is notified to the test center, the test apparatus 1 may access the test server 6, acquire the user information, and display a guide for confirming the user's name, address, telephone number, and the like on the touch panel 14 based on the acquired user information.

In addition, in a case where a sample is delivered, a container box for containing the sample may be provided in the test apparatus 1, the sample may be contained in the container box, the sample may be collected from the container box at a predetermined time, and the sample may be delivered to the test center.

Further, in the first embodiment, a postprandial hyperglycemic spike is used to determine an abnormal tendency in the blood glucose equivalent value. On the other hand, the present disclosure is not limited thereto. As an abnormal tendency determined by monitoring the blood glucose equivalent value, fasting hyperglycemia, hypoglycemia, nocturnal hyperglycemia, or the like may be determined. For example, in a case of hypoglycemia, the test candidates to be presented include a 5-hour tolerance test, a salivary cortisol test, a salivary cortisol+DHEA test, a delayed-type food allergy test, and organic acid urine measurement. The 5-hour tolerance test is an almost essential test as a test for hypoglycemia. The salivary cortisol test is a test to determine whether or not a person is in a fatigued state due to stress. The DHEA test is a test for dehydroepiandrosterone sulfate. The dehydroepiandrosterone sulfate is a type of male hormone. The delayed-type food allergy test is a test to determine severity of intestinal disorders. The organic acid urine measurement is a test to determine whether a fungus grows in an intestinal tract and produces toxins.

### Second Embodiment

In the first embodiment, an example in which the guide according to the test candidate for acquiring biological information associated with the blood glucose equivalent value according to the abnormal tendency in the blood glucose equivalent value is presented to the user has been described. On the other hand, the present disclosure is not limited thereto. For example, the technique of the present disclosure may be applied to an example in which a guide according to the test candidates for acquiring biological information required for diagnosing various diseases such as infectious diseases and cancers is presented to a user. It is known that some of the various diseases cause an abnormal tendency in physiological information such as a heart rate, a blood pressure, respiration, an electrocardiogram, maximum oxygen intake, arterial oxygen saturation, and a body temperature in a case of infection. In particular, in a case where the technique of the present embodiment can be used to determine abnormal tendencies in various physiological information before a user becomes aware of symptoms of the diseases and recommend the user to take various tests in a case where there is an abnormal tendency, it can contribute to early detection of diseases.

Hereinafter, an example in which a new coronavirus infection (COVID-19) is applied as a specific example of the diseases will be described. In the present embodiment, instead of the blood glucose equivalent value in the first embodiment, a heart rate is applied as the first biological information. It is assumed that a heart rate is acquired by a sensor included in the measurement device 3. Further, instead of the glucose, the urine glucose, the blood glucose value, HbA1c, glycoalbumin, and 1,5AG in the first embodiment, a test result related to the presence or absence of infection with new coronavirus is applied as second biological information. In the present embodiment, a repeated description for the same configuration and operation as those in the first embodiment will be omitted.

FIG. 33 is a schematic diagram illustrating an example of changes in heart rate variability (HRV), virus amount in a body, antibody amount in a body, and virus excretion amount of a patient who is infected with new coronavirus infection. In FIG. 33, the heart rate variability is illustrated by a thick solid line, the virus amount in a body is illustrated by a fine solid line, the antibody amount in a body is illustrated by a one-dotted line, and the virus excretion amount is illustrated by a broken line. In a horizontal axis of FIG. 33, an onset day of a disease when a user becomes aware of symptoms such as fever and cough is set as day 0, a day after the onset day of the disease is indicated as a day with plus, and a day before the onset day of the disease is indicated as a day with minus. The heart rate variability is a value representing variability in an R-R interval (RRI) for each heart rate. Specifically, the heart rate variability is represented by a standard deviation of the R-R interval (SDNN) for a predetermined period and/or a root mean square of successive two R-R interval differences (rMSSD).

As illustrated in FIG. 33, in a case of the new coronavirus infection, a virus amount in a body begins to increase rapidly about 5 days before the onset day, and this results in an abnormal tendency such as a decrease in heart rate variability. The reason is as follows. In a case of the heart rate variability of an uninfected person, the heart rate increases during exercise or in a tension state, and the heart rate decreases in a relaxation state. On the other hand, in a case of the heart rate variability of an infected person, followability of the heart rate is poor and a degree of increase/decrease in the heart rate is small. Thereafter, near the onset day, the virus amount in the body reaches a maximum value and the heart rate variability reaches a minimum value. Further, about 10 days after the onset day, the antibody amount in the body begins to increase and the virus amount in the body begins to decrease, and thus the heart rate variability gradually returns to normal.

In the test related to new coronavirus infection, as a sample that can be collected by the user himself/herself, nasal swab, saliva, and blood are used (details will be described). The analysis apparatus 18 according to the present embodiment obtains, as second biological information, a test result related to the presence or absence of the new coronavirus infection by analyzing the user's nasal swab, saliva, and blood. Thus, the analysis apparatus 18 includes a mechanism for collecting the user's nasal swab, saliva, and blood, a mechanism for acquiring the nasal swab, saliva, and blood from the test kit 20A to be described, and the like.

The container case 20 according to the present embodiment contains plural test kits 20A for collecting the user's nasal swab, saliva, and blood. The test kit 20A includes, for example, a cotton swab for collecting nasal swab, a container for collecting saliva, and a blood sampling device.

The acquisition unit 51 acquires the monitoring result of the blood glucose equivalent value transmitted from the communication I/F 35 of the measurement device 3 by receiving the monitoring result of the heart rate by the communication I/F 15. In the present embodiment, for example, the monitoring result of the heart rate measured in the last 24 hours is acquired.

The determination unit 52 determines whether or not there is an abnormal tendency in the heart rate based on the monitoring result of the heart rate acquired by the acquisition unit 51. Specifically, the determination unit 52 obtains heart rate variability from the monitoring result of the heart rate transmitted from the measurement device 3, and determines that there is an abnormal tendency in the heart rate in a case where there is a situation in which the heart rate variability is equal to or smaller than a predetermined threshold value Th11. Further, the determination unit 52 determines that a degree of the abnormal tendency in the heart rate is large in a case where the heart rate variability is equal to or smaller than a threshold value Th12 (here, Th12<Th11), and determines that a degree of the abnormal tendency in the heart rate is small in a case where the heart rate variability is equal to or smaller than the threshold value Th11 and larger than the threshold value Th12.

Further, the determination unit 52 may determine the abnormal tendency in the heart rate by using a representative value such as an average value and a median value of the heart rate variability per unit time for each predetermined period (for example, the last 24 hours). This is because it is preferable that a temporary increase in the heart rate variability during exercise and in a tension state is not considered as an abnormal tendency.

Further, in a case where the heart rate variability is in an increase state (equal to or larger than the threshold value Th11), the determination unit 52 determines whether or not the increase is a temporary increase during exercise, in a tension state, or the like. In a case where it is determined that the increase is a temporary increase, the determination unit 52 may determine whether or not there is an abnormal tendency in the heart rate during a period other than a period for which the heart rate variability increases. Whether or not the heart rate variability is a temporary increase may be determined by comparing the heart rate variability with a temporary increase tendency of the heart rate variability in the previous same situation. Specifically, for example, a temporary increase pattern of the heart rate variability is stored in advance in the storage 13, and the determination unit 52 may determine whether or not the heart rate variability is a temporary increase by comparing the stored increase pattern with the monitoring result of the heart rate.

In a case where the determination unit 52 determines that there is an abnormal tendency in the heart rate, the decision unit 53 decides a test candidate to be recommended for the user.

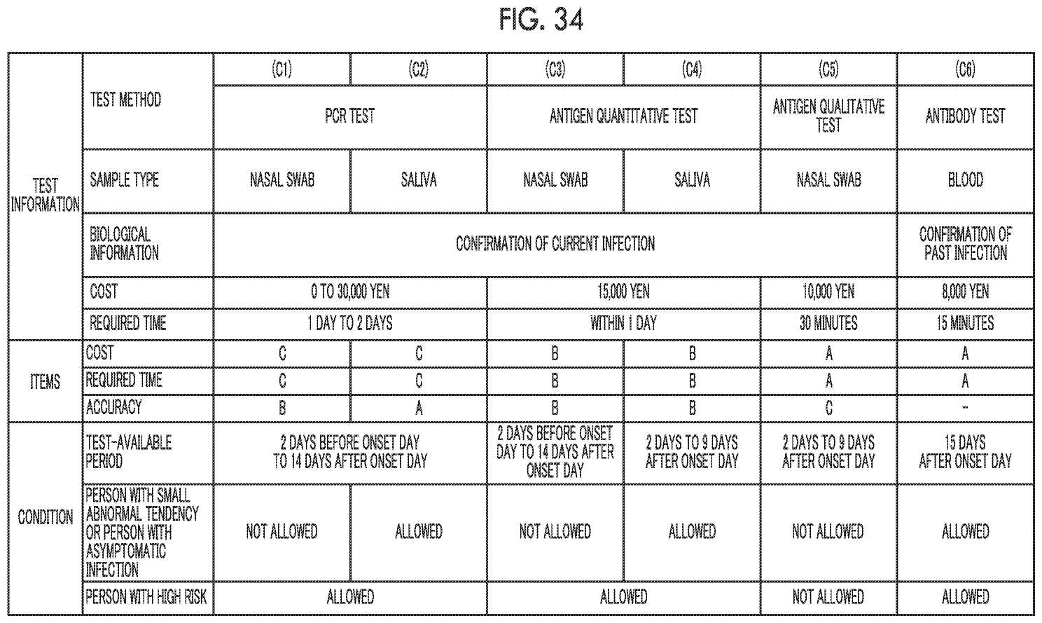

A specific example of a method of deciding a test candidate by the decision unit 53 will be described with reference to FIG. 34. FIG. 34 is a table illustrating test methods related to the new coronavirus infection. In FIG. 34, for 6-type test methods (C1) to (C6), a sample type, biological information to be acquired, a cost, and a required time are illustrated as test information. The table illustrated in FIG. 34 is stored, as a table, for example, in the storage 13. The content of FIG. 34 is an example, and the test information, an evaluation and a condition of each item may be updated as appropriate according to an epidemic situation and a treatment situation of the new coronavirus infection, a progress state of the test method, and the like.

The test methods (C1) and (C2) are polymerase chain reaction (PCR) tests in which the samples are nasal swab and saliva. The test methods (C3) and (C4) are antigen quantitative tests in which the samples are nasal swab and saliva. The test method (C5) is an antigen qualitative test in which the sample is nasal swab. In the test methods (C1) to (C5), a test result as to whether or not the patient is currently infected with the new coronavirus infection can be acquired as the second biological information. The test method (C6) is an antibody test using blood as a sample. In the test method (C6), a test result as to whether or not the patient is previously infected with the new coronavirus infection can be acquired as the second biological information. The decision unit 53 selectively decides the test candidate from plural the test methods (C1) to (C6) including designation of a type of the sample and for acquiring the second biological information from the sample.

FIG. 34 illustrates various items serving as indexes in a case where the user selects the presented test candidate. The definitions of the various items and the evaluation methods are the same as those in the first embodiment, and thus a description thereof will be omitted. On the other hand, in a case of accuracy, as a degree to which a disease-positive person can be detected as positive (so-called sensitivity) is higher, a degree of the accuracy is higher.

FIG. 34 illustrates various conditions in a case where the decision unit 53 decides the test candidate. Specifically, conditions related to a test-available period and test suitability for a person with a small abnormal tendency, a person with asymptomatic infection, and a person with a high risk are illustrated. The “test-available period” indicates a period for which a degree to which a disease-positive person can be detected as positive (so-called sensitivity) in a case where a test is performed for the disease-positive person is relatively high and a test result is reliable. The “person with a small abnormal tendency” is a person determined by the determination unit 52 as having a small abnormal tendency. The “person with asymptomatic infection” means a person whose symptoms such as fever and cough are not developed. For the person with a small abnormal tendency and the person with asymptomatic infection, it is considered that a highly-sensitive test method should be adopted in order to prevent an error in determination due to false negatives, and as a result, a test method with a relatively low sensitivity is not allowed. The “person with a high risk” is a person who has a risk factor such as a serious condition and a high treatment difficulty, and is, for example, an elderly person, a person having an underlying disease, a pregnant woman, or the like. For the person with a high risk, it is considered that a test should be performed with higher accuracy in order to quickly perform a proper treatment, and as a result, a test method with a relatively low sensitivity is not allowed.

The decision unit 53 decides, as a test candidate, a test method suitable for various conditions among the test methods (C1) to (C6) by referring to the table of FIG. 34. As illustrated in FIG. 34, in a case of new coronavirus infection, the test-available period is determined based on the onset day. Firstly, the decision unit 53 estimates the onset day based on the heart rate variability, and decides the test candidate according to whether the current time is included in the test-available period based on the estimated onset day. For example, in a case where it is estimated that the current time is two days before the onset day, the decision unit 53 decides the test methods (C1) to (C3) as test candidates. In addition, for example, in a case where it is estimated that the current time is four days after the onset day, the decision unit 53 decides the test methods (C1) to (C5) as test candidates. Further, for example, in a case where it is estimated that the current time is 15 days or more after the onset day, the decision unit 53 decides the test method (C6) as a test candidate.

As a method for estimating the onset day, based on, for example, a fact that there is a time lag of about 5 days from a time when the abnormal tendency in the heart rate variability starts to be observed to the onset day (refer to FIG. 33), a method of estimating the onset day to about 5 days after a time when the determination unit 52 determines that there is an abnormal tendency in the heart rate may be used. Further, for example, a method of estimating the onset day using a learning model may be used, the learning model being learned by using, as learning data, a pair of transition data of the heart rate variability of a patient who is infected with new coronavirus infection and an actual onset day, and being a model that receives the transition data of the heart rate variability and outputs the estimated onset day.

Secondly, the decision unit 53 decides the test candidate according to the degree of the abnormal tendency in the heart rate that is determined by the determination unit 52. For example, in a case where the determination unit 52 determines that the abnormal tendency is small, the decision unit 53 decides the test methods (C2), (C4), and (C6) as test candidates. Further, the decision unit 53 decides the test candidates in the same manner even in a case where the user is a person with asymptomatic infection. On the other hand, in a case where the determination unit 52 determines that the abnormal tendency is large, the decision unit 53 decides the test methods (C1) to (C6) as test candidates.

Thirdly, the decision unit 53 decides the test candidate according to a risk factor that the user has. For example, in a case where the user is a person with a high risk, the decision unit 53 decides the test methods (C1) to (C4) and (C6) as test candidates. On the other hand, in a case where the user is not a person with a high risk, the decision unit 53 decides the test methods (C1) to (C6) as test candidates.

According to the first to third decisions, the decision unit 53 decides a final test candidate. For example, in a case where, the current time is four days after the onset day, the determination unit 52 determines that the abnormal tendency is large, and the user is a person with symptomatic infection and is not a person with a high risk, the decision unit 53 decides the test methods (C1) to (C5) as test candidates. Further, for example, in a case where, the current time is four days after the onset day, the determination unit 52 determines that the abnormal tendency is large, and the user is a person with asymptomatic infection and is a person with a high risk, the decision unit 53 decides the test methods (C2) and (C4) as test candidates.

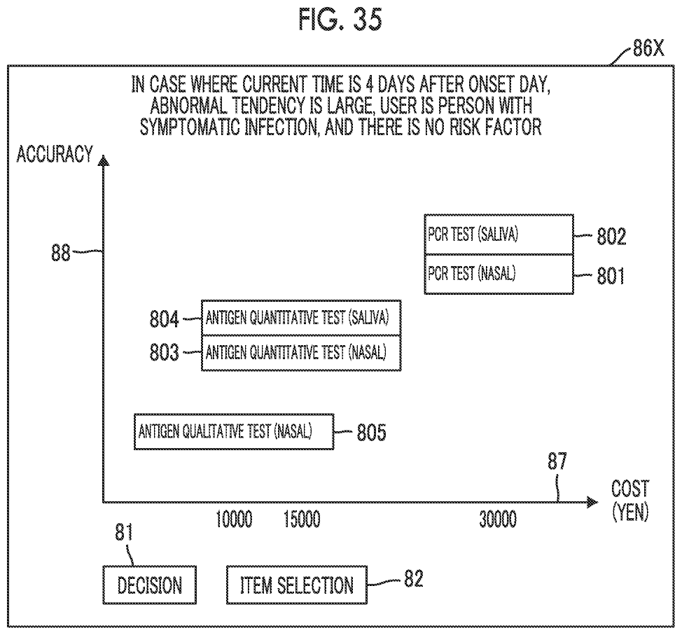

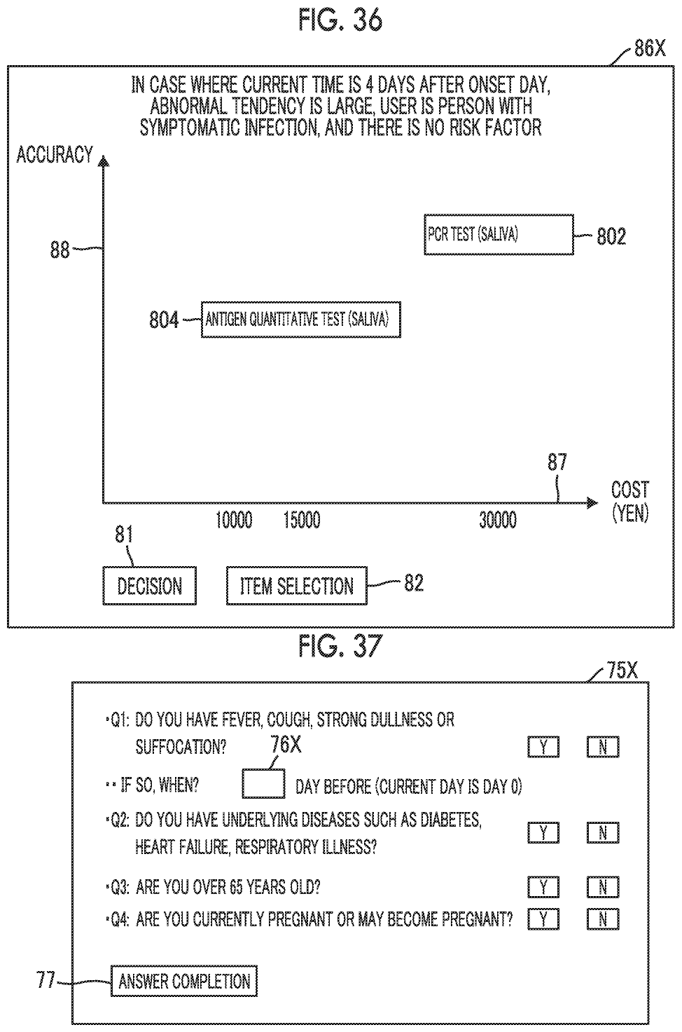

The presentation unit 54 presents, to the user, a test candidate decided by the decision unit 53. FIG. 35 and FIG. 36 are diagrams illustrating an example of a presentation screen for presenting the test candidates to the user in a display form in which the test candidate icons are displayed on the scale in a case where two items of “cost” and “accuracy” are selected. As illustrated in FIG. 35 and FIG. 36, on the presentation screen 86X, a scale 87 representing the cost and a scale 88 representing the accuracy are displayed so as to be perpendicular to each other, and test candidate icons 801 to 805 are displayed so as to be arranged in a two-dimensional shape according to the cost and the accuracy. The icons 801 to 805 respectively correspond to the test methods (C1) to (C5).

FIG. 35 illustrates a presentation screen in a case where, the current time is four days after the onset day, the determination unit 52 determines that the abnormal tendency is large, and the user is a person with symptomatic infection and is not a person with a high risk. FIG. 36 is a presentation screen in a case where, the current time is four days after the onset day, the determination unit 52 determines that the abnormal tendency is large, and the user is a person with asymptomatic infection and is a person with a high risk. The user can recognize the cost and the accuracy for the presented test candidate at a glance based on the presentation screen 86X. Thereby, it is possible to easily compare the test candidates in consideration of both the cost and the accuracy.

The presentation unit 54 receives selection of the presented test candidates by the user. Thereafter, the presentation unit 54 presents, to the user, a guide according to the selected test candidate by using the touch panel 14. For example, the presentation unit 54 presents, to the user, a guide for guiding the user to remove the test kit 20A for self collecting any one of nasal swab, saliva, or blood from the container case 20 according to the selected test candidate. In addition, the presentation unit 54 presents, to the user, a guide on the self collecting method. Information on the guide may be stored in the storage 13. The presentation unit 54 may read the guide according to the test candidate selected by the user from the storage 13, and display the read guide on the touch panel 14. The user follows the guide and performs self collecting of any one of nasal swab, saliva, or blood.

Further, the presentation unit 54 may present, as a guide, support information for supporting execution of a test of the selected test candidate, the support information being transmitted in real time from a remote location. The test apparatus 1 may further include a camera that captures an image of the user. Further, the presentation unit 54 may transmit, to the remote location, a moving image obtained by capturing a state where the user performs the test of the selected test candidate by the camera. Specifically, in a case where a computer owned by a medical staff and the test apparatus 1 are connected to each other via a network, the medical staff at a remote location may present, to the user, a guide on a self collecting method in real time. In this case, the camera included in the test apparatus 1 may obtain a moving image by capturing a state where the user performs self collecting of nasal swab, saliva, or blood, and transmit the moving image to the computer of the medical staff at a remote location. Further, the test apparatus 1 may further include a speaker, and may reproduce a voice guide from the medical staff by using the speaker.

Further, in a state where the current time is three days before the onset day, in a case where the decision unit 53 decides that any of the test methods is not suitable, the presentation unit 54 may present a guide indicating a fact that any of the test methods is not suitable. For example, a guide indicating “There is an abnormal tendency in the heart rate. But, at this time, an accurate result cannot be obtained by a test. Please continue to monitor your heart rate.” is presented. Further, in a case where the determination unit 52 determines that there is no abnormal tendency in the heart rate, the presentation unit 54 may present a guide indicating a fact that there is no abnormal tendency in the heart rate. For example, a guide indicating “Currently, there is no abnormal tendency in the heart rate. Please continue to take measures against infection.” is presented.

As described above, in the present embodiment, in a case where there is an abnormal tendency in the monitoring result of the heart rate of the user, the test candidates for acquiring a test result related to the presence or absence of new coronavirus infection associated with the heart rate are presented to the user, and a guide according to the test candidate selected by the user is presented to the user. Therefore, the user can perform a test according to the monitoring result of the heart rate by using the test apparatus according to the present embodiment. In particular, in a case of new coronavirus infection, the virus excretion amount of a patient who is infected with new coronavirus infection, that is, infectivity to others reaches a peak near the onset day (refer to FIG. 33). Thus, by determining the abnormal tendency in the heart rate before the onset day and presenting the user to take a test, it is possible to contribute to prevention of spread of infection.

In the second embodiment, an example in which the determination unit 52 determines the abnormal tendency in the heart rate based on the heart rate variability has been described. On the other hand, the present disclosure is not limited thereto. The determination unit 52 may determine that there is an abnormal tendency in the heart rate in a case where, instead of the heart rate variability, the heart rate value is equal to or higher than a predetermined threshold value for a predetermined period (for example, three days). According to the example, it is possible to contribute to early detection of a disease in which the heart rate is maintained in a high state at the time of infection. The determination unit 52 may determine that there is an abnormal tendency in the heart rate in a case where, instead of the heart rate variability, a temporal variation in the heart rate value (a difference between a maximum heart rate and a minimum heart rate for a predetermined period (for example, 3 days)) is equal to or larger than a predetermined threshold value. According to the example, it can contribute to early detection of a disease in which the heart rate sharply increases at the time of infection.

Further, in the second embodiment, an example in which the heart rate is applied as the first biological information has been described. On the other hand, the present disclosure is not limited thereto. In the second embodiment, as the first biological information, at least one piece of physiological information such as a heart rate, a blood pressure, respiration, an electrocardiogram, maximum oxygen intake, arterial oxygen saturation, and a body temperature may be applied. The physiological information is acquired by, for example, a sensor included in a wearable terminal such as a smart watch.

Further, in the second embodiment, an example in which a test result related to the presence or absence of infection with new coronavirus infection is applied as the second biological information has been described. On the other hand, the present disclosure is not limited thereto. In the second embodiment, a diagnosis result of a disease of the user may be applied as the second biological information associated with physiological information. As the “diagnosis result of a disease”, for example, an analysis result of components and a detection result of pathogens such as a virus and a bacteria using, as a sample, body fluids such as blood, urine, feces, nasopharyngeal swab, nasal swab, and saliva of the user may be applied. Further, for example, a reading result of an image, which is obtained by imaging an organ as a sample such as a stomach, a large intestine, a lung, a uterus, and a breast of the user by using a method such as computed tomography (CT), magnetic resonance imaging (MRI), and ultrasound, may be applied.

Further, in the second embodiment, diagnosis results of plural different types of diseases may be applied as the second biological information associated with physiological information. For example, both of a test related to new coronavirus infection and a test related to influenza virus infection may be presented as test candidates. In addition, a guide indicating that the user can take tests related to the plurality of different types of diseases at the same time may be presented. In such a case, diagnosis for various diseases can be recommended, and thus it is possible to contribute to early detection of diseases.

In the second embodiment, the onset day and information on whether the user corresponds to a person with asymptomatic infection or a person with a high risk may be input from the user via, for example, the touch panel 14. FIG. 37 is a diagram illustrating a question presentation screen for inputting an onset day and information on a user. As illustrated in FIG. 37, four questions Q1 to Q4 are displayed on the question presentation screen 75X. The user answers YES or NO to the questions Q1 to Q4. In a case where an answer to the question Q1 is YES, the user inputs an onset day in an input box 76X. Thereby, the decision unit 53 specifies the onset day. In a case where an answer to the question Q1 is NO, the decision unit 53 specifies that the user is a person with asymptomatic infection. In a case where an answer to at least one of the questions Q2 to Q4 is YES, the decision unit 53 specifies that the user is a person with a high risk. In a case where the user selects an answer completion button 77 after answering, the decision unit 53 decides the test candidate according to answer results of the four questions Q1 to Q4.

Further, in the second embodiment, based on a fact that there is a time lag of about 5 days from a time when the abnormal tendency in the heart rate starts to be observed to the onset day (refer to FIG. 33), the decision unit 53 may estimate whether or not the user is a person with asymptomatic infection. For example, in a case where the current time is two days after a time when the abnormal tendency in the heart rate starts to be observed, the decision unit 53 may estimate that the user is a person with asymptomatic infection whose symptoms are not yet developed. Further, for example, in a case where a body temperature is measured by the measurement device 3, the decision unit 53 may determine the presence or absence of fever, and in a case where there is no fever, may estimate that the user is a person with asymptomatic infection.

Further, in the second embodiment, an example in which the decision unit 53 estimates the onset day based on a fact that the test-available period of new coronavirus infection is determined based on the onset day has been described. On the other hand, the present disclosure is not limited thereto. For example, in a case where influenza virus infection is tested, a time from an infection to an onset is as short as 1 day to 2 days, and as a result, a difference between the day when the abnormal tendency in the first biological information starts to be observed and the onset day is small. Thus, significance of estimating the onset day is small. Further, for example, in a case where various lifestyle-related diseases are tested, it is difficult to estimate an accurate onset day. On the other hand, even in these diseases, appropriate test methods may differ according to an elapsed time from a time when the abnormal tendency starts to be observed. The decision unit 53 may decide the test candidate according to an elapsed time from a time when it is determined that there is the abnormal tendency in the first biological information to a current time.

Further, in the second embodiment, as a condition for determining the test candidate, a condition as to whether or not the user is a close contact person may be applied. In a case where the user is a close contact person, possibility that the user is detected as a disease-positive person is relatively high. Thus, it is considered that a highly-sensitive test method should be adopted in order to prevent an error in determination due to false negatives. Therefore, a test method having a relatively low sensitivity may not be allowed. Whether or not the user is a close contact person may be detected by, for example, a known application for new coronavirus contact confirmation.

Further, in the second embodiment, after a test related to the presence or absence of infection with the new coronavirus infection is performed, the test result is fed-back. In this case, the test apparatus 1 may determine whether to continue monitoring of the heart rate. For example, in a case where the determination unit 52 determines that there is an abnormal tendency in the heart rate, a test for new coronavirus infection is performed. On the other hand, in spite of the test, in a case where the test result is negative, it may be determined that monitoring of the heart rate is continued. In particular, in a case where the user corresponds to any one of a person with a small abnormal tendency, a person with asymptomatic infection, a person with a high risk, or a close contact person, it is considered that there is a high risk of an error in determination and a sudden change in physical condition due to false negatives. Thus, preferably, it is determined that monitoring of the heart rate is continued.

Specifically, the acquisition unit 51 acquires a test result related to the presence or absence of infection with new coronavirus infection. The test result may be transmitted from the analysis apparatus 18 to the acquisition unit 51, or may be input from the user via the touch panel 14. In a case where the acquired test result is negative, the presentation unit 54 determines that monitoring of the heart rate is continued, and presents a guide indicating a fact that monitoring of the heart rate is continued to the user. In a case where monitoring of the heart rate is continued, the determination unit 52 determines whether or not the abnormal tendency in the heart rate becomes more remarkable and whether or not the abnormal tendency in the heart rate continues. In a case where the determination unit 52 determines that the abnormal tendency in the heart rate becomes more remarkable or that the abnormal tendency in the heart rate continues, the decision unit 53 decides test candidates to be recommended again for the user.

Further, in the second embodiment, the test methods (C1) to (C6) of FIG. 34 do not include a PCR test using a nasopharyngeal swab as a sample, an antigen quantitative test, and an antigen qualitative test, which are generally used as a test method for new coronavirus infection. This is because it is difficult to perform self collecting of nasopharyngeal swab. On the other hand, for example, in a case where a medical staff is present in the vicinity of the test apparatus 1 and the medical staff collects the user's nasopharyngeal swab, a PCR test using a nasopharyngeal swab as a sample, an antigen quantitative test, and an antigen qualitative test can be set as test candidates.

Further, in the second embodiment, in any of the test methods (C1) to (C6) of FIG. 34, a guide for guiding the user to deliver the sample collected by the test kit 20A to the test center may be presented to the user, and the test of the sample may be performed at the test center. In this case, the presentation unit 54 may transmit heart rate data of the user and the test candidate decided by the decision unit 53 to the test center. According to the example, it is possible to assist a medical staff in the test center in determining whether the test method selected by the user is appropriate. In this case, the test apparatus 1 may not include the analysis apparatus 18.

Further, in the embodiment, the presentation unit 54 presents the test candidates by displaying the test candidates on the touch panel 14. On the other hand, the present disclosure is not limited thereto. The presentation unit 54 may present a test candidate to the user by voice.

Further, in the embodiment, the test apparatus 1 may include a mechanism for cleaning a portion that the user may come into contact with, such as the touch panel 14, the housing of the test apparatus 1, and the vicinity of the location of the test apparatus 1, each time the user completes use of the test apparatus 1. Examples of the cleaning include wiping by using a cloth or the like, spraying of a disinfectant solution, irradiation with ultraviolet rays, and the like. According to the example, even in a case where nasal swab, saliva, blood, or the like obtained by self collecting by the user adheres to the portion, the test apparatus 1 is not in an unsanitary state, and it is possible to prevent spread of infectious diseases.

Further, in the embodiment, the monitoring result of the first biological information measured by the measurement devices 3 and 4 is transmitted to the test apparatus 1. On the other hand, the present disclosure is not limited thereto. The first biological information measured by the measurement devices 3 and 4 may be transmitted to the mobile terminal 2, and the monitoring result of the first biological information may be transmitted from the mobile terminal 2 to the test apparatus 1.

Further, in the embodiment, the monitoring result of the first biological information measured by the measurement devices 3 and 4 is transmitted to the test apparatus 1, and the determination unit 52 determines the abnormal tendency in the first biological information based on the monitoring result of the first biological information. On the other hand, the present disclosure is not limited thereto. The measurement devices 3 and 4 may determine the abnormal tendency in the first biological information based on the monitoring result of the first biological information, and transmit the determination result to the test apparatus 1. In this case, the measurement program 32 performs processing of determining the abnormal tendency. Further, in the test apparatus 1, without performing determination by the determination unit 52, the decision unit 53 decides the test candidates based on the received determination result.

Further, the first biological information measured by the measurement devices 3 and 4 may be transmitted to the mobile terminal 2, and the mobile terminal 2 may determine the abnormal tendency in the first biological information based on the monitoring result of the first biological information. The determination result may be transmitted from the mobile terminal 2 to the test apparatus 1. In this case, the analysis program 22 performs processing of determining the abnormal tendency. Further, in the test apparatus 1, without performing determination by the determination unit 52, the decision unit 53 may decide the test candidates based on the received determination result.

Further, in the embodiments, for example, as a hardware structure of processing units that execute various processing, such as the acquisition unit 51, the determination unit 52, the decision unit 53, and the presentation unit 54, the following various processors are may be used. The various processors include, as described above, a CPU, which is a general-purpose processor that functions as various processing units by executing software (program), and a dedicated electric circuit, which is a processor having a circuit configuration specifically designed to execute a specific processing, such as a programmable logic device (PLD) or an application specific integrated circuit (ASIC) that is a processor of which the circuit configuration may be changed after manufacturing such as a field programmable gate array (FPGA).

One processing unit may be configured by one of these various processors, or may be configured by a combination of two or more processors having the same type or different types (for example, a combination of plural FPGAs or a combination of a CPU and an FPGA). Further, the plurality of processing units may be configured by one processor.

As an example in which the plurality of processing units are configured by one processor, firstly, as represented by a computer such as a client and a server, a form in which one processor is configured by a combination of one or more CPUs and software and the processor functions as the plurality of processing units may be adopted. Secondly, as represented by a system on chip (SoC) or the like, a form in which a processor that realizes the function of the entire system including the plurality of processing units by one integrated circuit (IC) chip is used may be adopted. As described above, the various processing units are configured by using one or more various processors as a hardware structure.

Further, as the hardware structure of the various processors, more specifically, an electric circuit (circuitry) in which circuit elements such as semiconductor elements are combined may be used.

What is claimed is:

1. A test apparatus comprising at least one processor, wherein the processor is configured to
acquire a determination result as to whether or not there is an abnormal tendency in first biological information, the determination result being determined based on a monitoring result of the first biological information of a user,
determine, as a test candidate to be recommended for the user, at least one test candidate for acquiring second biological information associated with the first biological information in a case where the determination result corresponds to the abnormal tendency,
present the determined test candidate to the user,
receive selection of the presented test candidate, and
present a guide according to the selected test candidate to the user.

2. The test apparatus according to claim 1, wherein the processor is configured to
present the test candidate for acquiring the second biological information by collecting a sample of the user,
present, to the user, a guide for collecting the sample in a case where the test candidate is selected, and
acquire the second biological information based on the sample collected by the user.

3. The test apparatus according to claim 2, wherein the processor is configured to
present the second biological information to the user.

4. The test apparatus according to claim 2, further comprising an analysis apparatus that obtains the second biological information by analyzing the collected sample.

5. The test apparatus according to claim 1, wherein the processor is configured to
present the test candidate for acquiring the second biological information by delivering a sample of the user to an external test agency, and
present, to the user, a guide for a procedure of delivering the first biological information of the user to the user in a case where the test candidate is selected.

6. The test apparatus according to claim 1, further comprising a container case that contains a test kit required for performing a test according to the test candidate to be presented.

7. The test apparatus according to claim 1, wherein the test candidate includes a test candidate in which the second biological information is acquired with accuracy higher than accuracy of the first biological information.

8. The test apparatus according to claim 1, wherein the processor is configured to
receive designation of at least one item serving as an index in a case where the user selects the presented test candidate, and
present the test candidate in a display form according to the designated item.

9. The test apparatus according to claim 8, wherein the processor is configured to
present, to the user, a question for determining a display position of the test candidate on at least one scale according to the designated at least one item,
determine the display position of the test candidate on the scale according to an answer to the question, and
present, to the user, the test candidate in a display form in which an icon representing the test candidate is displayed at the determined display position on the scale.

10. The test apparatus according to claim 9, wherein the processor is configured to
receive additional designation of the item by the user after the icon representing the test candidate is displayed, and
update the display form of the icon representing the test candidate in a case where designation of the item is added.

11. The test apparatus according to claim 10, wherein the processor is configured to
repeatedly perform receiving of additional designation of the item, presenting of a question according to the added item, and updating of the display form of the test candidate until the test candidate is selected by the user.

12. The test apparatus according to claim 8, wherein the item includes at least one of a cost required for a test, a sense of stability in a case where a sample is collected, a degree of restraint in a test, invasiveness or non-invasiveness, a time required to obtain a test result, whether to include a risk check for various diseases, or test accuracy.

13. The test apparatus according to claim 1, wherein the processor is configured to
selectively determine the test candidate from a plurality of test methods including designation of a type of a sample and used for acquiring the second biological information from the sample.

14. The test apparatus according to claim 1, wherein the processor is configured to
determine the test candidate according to a degree of the abnormal tendency.

15. The test apparatus according to claim 1, wherein the processor is configured to
determine the test candidate according to an elapsed time from a time when it is determined that there is the abnormal tendency in the first biological information to a current time.

16. The test apparatus according to claim 1, wherein the processor is configured to
determine the test candidate according to a risk factor that the user has.

17. The test apparatus according to claim 1, wherein the processor is configured to
present, as the guide, support information for supporting execution of a test of the selected test candidate, the support information being transmitted in real time from a remote location.

18. The test apparatus according to claim 17, further comprising a camera that captures an image of the user,
wherein the processor is configured to
transmit, to the remote location, a moving image obtained by capturing a state where the user performs the test of the selected test candidate by the camera.

19. The test apparatus according to claim 1, wherein the processor is configured to
present, to the user, statistical information of the test candidate which is previously selected.

20. The test apparatus according to claim 1, wherein the first biological information is a blood glucose equivalent value that correlates with a blood glucose value, and
the second biological information includes at least one of a blood glucose value or HbA1c.

21. The test apparatus according to claim 1, wherein the abnormal tendency is determined based on a postprandial hyperglycemic spike.

22. The test apparatus according to claim 21, wherein the processor is configured to
determine the test candidate based on meal content of the user.

23. The test apparatus according to claim 21, wherein the processor is configured to
determine the test candidate based on a meal time of the user.

24. The test apparatus according to claim 23, wherein the processor is configured to
present the test candidate including an action of drinking a glucose drink in a case where a predetermined time has elapsed from a time after meal based on the meal time.

25. The test apparatus according to claim 1, wherein the first biological information is at least one of a heart rate, a blood pressure, respiration, an electrocardiogram, maximum oxygen intake, arterial oxygen saturation, or a body temperature, and
the second biological information is a diagnosis result of a disease of the user.

26. The test apparatus according to claim 1, wherein the processor is configured to
notify the user of a fact that there is no abnormal tendency in the first biological information in a case where a determination result indicating the fact is acquired.

27. The test apparatus according to claim 1, wherein the first biological information is monitored by a measurement device that the user wears,
the processor is configured to
acquire the monitoring result of the first biological information from the measurement device, and
acquire the determination result by determining whether or not there is the abnormal tendency in the first biological information based on the acquired monitoring result of the first biological information.

28. The test apparatus according to claim 1, wherein the determination result is obtained by a measurement device attached to the user, and
the processor is configured to
acquire the determination result from the measurement device.

29. The test apparatus according to claim 27, wherein the measurement device is a wearable measurement device.

30. The test apparatus according to claim 27, wherein the processor is configured to
perform a notification for urging the user to wear the measurement device for a longer time in a case where whether or not there is the abnormal tendency in the first biological information is not determined.

31. The test apparatus according to claim 1, wherein the processor is configured to
acquire the second biological information, and
determine whether to continue monitoring of the first biological information according to the acquired second biological information.

32. A test method comprising:
acquiring a determination result as to whether or not there is an abnormal tendency in first biological information, the determination result being determined based on a monitoring result of the first biological information of a user;
determining, as a test candidate to be recommended for the user, at least one test candidate for acquiring second biological information associated with the first biological information in a case where the determination result corresponds to the abnormal tendency;
presenting the determined test candidate to the user;
receiving selection of the presented test candidate; and
presenting a guide according to the selected test candidate to the user.

33. A non-transitory computer-readable storage medium storing a test program causing a computer to execute:
a procedure of acquiring a determination result as to whether or not there is an abnormal tendency in first biological information, the determination result being determined based on a monitoring result of the first biological information of a user;
a procedure of determining, as a test candidate to be recommended for the user, at least one test candidate for acquiring second biological information associated with the first biological information in a case where the determination result corresponds to the abnormal tendency;
a procedure of presenting the determined test candidate to the user;
a procedure of receiving selection of the presented test candidate; and
a procedure of presenting a guide according to the selected test candidate to the user.
